(19) United States
(12) Patent Application Publication
(10) Pub. No.: US 2025/0164353 A1
(43) Pub. Date: May 22, 2025

(54) SAMPLE COLLECTION AND DISPENSING DEVICES AND METHODS

(71) Applicant: Nicoya Lifesciences Inc., Kitchener (CA)

(72) Inventor: Sebastian VON DER ECKEN, Concord (CA)

(21) Appl. No.: 18/836,688

(22) PCT Filed: Feb. 6, 2023
(86) PCT No.: PCT/CA2023/050150

§ 371 Date: Aug. 7, 2024

Related U.S. Application Data
(60) Provisional application No. 63/400,190, filed on Aug. 23, 2022.
(60) Provisional application No. 63/307,199, filed on Feb. 7, 2022.

(51) Int. Cl. G01N 1/14; B01L 3/00

(52) U.S. Cl. CPC G01N 1/14; B01L 3/5023; B01L 2300/069; B01L 2400/0478; G01N 2001/1427; G01N 2001/149

(57) ABSTRACT

Sample collection and dispensing devices and methods for collecting and dispensing a sample fluid using the devices. The devices and methods may provide a plunger for holding a sample collection sponge and a flexible and/or squeezable barrel having an open end for receiving the plunger and sample collection sponge and a closed end. The plunger may include one or more flow channels operable to dispense a fluid from the barrel. A sample volume is collected using the sample collection sponge. the plunger is inserted into the barrel, and the sponge is compressed against the closed end of the barrel. causing sample liquid to flow from the sponge into the one or more flow channels in the plunger. Sample fluid may be stored in the device. The devices and methods provide a way of manually collecting, storing, and dispensing a precise amount of a sample liquid. such as saliva.

### CROSS REFERENCE TO OTHER APPLICATIONS

This application claims the benefit of U.S. Provisional Application No. 63/400,190,filed Aug. 23, 2022, and U.S. Provisional Application No. 63/307,199, filed Feb. 7, 2022, which applications are incorporated herein in their entirety by reference.

### FIELD OF THE DISCLOSURE

The subject matter relates generally to the processing of biological materials and more particularly to sample collection and dispensing devices and methods.

### BACKGROUND

Microfluidic systems and devices are used in a variety of applications to manipulate, process and/or analyze biological materials. For example, microfluidic systems and devices are used in point-of-care (POC) applications. One challenge in POC testing is to combine the precise concentrations of samples and reagents. Pipetting is the standard in every laboratory environment. Laboratory workers have been using pipettes practically like an extension of their hand. This makes pipetting extremely desirable when, for example, flexibility in workflows, reagent types, and/or reagent volumes is necessary. This is not always the case in POC applications, such as home testing, where untrained users have shown difficulty in using a pipette and, in particular, in collecting and then dispensing a precise amount of sample. Unfortunately, current sample collection and dispensing devices are not directed toward non-expert users.

### SUMMARY

In some aspects, the present invention is directed to a sample collection and dispensing device, the device comprising: (a) a plunger, the plunger having a linear axis, a first end, a second end and a first flow channel running along the linear axis of the plunger; (b) a sample collection sponge, wherein the sample collection sponge is attached to the second end of the plunger; and (c) a cylinder-shaped barrel, wherein the cylinder-shaped barrel comprises a first open end for receiving the plunger and a second closed end, and wherein the cylinder-shaped barrel comprises a flexible side wall; and wherein the sponge is operable to collect a sample fluid; wherein the cylinder-shaped barrel is capable of holding and storing the sample fluid; and wherein the first flow channel is operable to dispense a fluid from the cylinder-shaped fluid reservoir.

In one embodiment, the first flow channel comprises a first end and a second end, wherein the first end comprises an open dispensing outlet and the second end is connected to a first exit hole in the side of the plunger. In some embodiments, the sample collection and dispensing device further comprises a second flow channel, and wherein the second flow channel connects the second end of the plunger to a second exit hole in the side of the plunger. In one embodiment, the second exit hole comprises a T-shaped exit hole having two openings on opposite sides of the plunger.

In one embodiment, the cylinder-shaped barrel has an internal volume of from about 20 μL to about 2000 μL.

In some embodiments, the sample collection and dispensing device further comprise a removable cap, and wherein the removable cap is attached to the first end of the plunger to cap or close the dispensing outlet of the first flow channel.

In one embodiment, the plunger further comprises a circular-shaped plate at the dispensing end of the plunger and wherein the plate has a plate diameter, wherein the plate diameter is the same as, or larger than a diameter of the cylinder-shaped barrel. In one embodiment, the plate is operable to cover the cylinder-shaped barrel.

In some embodiments, the plunger and the cylinder-shaped barrel further comprise threads and wherein the threads of the plunder and capable of engaging the threads of the cylinder-shaped barrel. In one embodiment, the threads on the plunger are on the underside of the circular-shaped plate and the threads on the cylinder-shaped barrel are on the inner wall of the cylinder-shaped barrel.

In some embodiments, the plate further comprises a seal or a gasket. In one embodiment, the seal or gasket is operable to close the open end of the cylinder-shaped barrel and optionally wherein the seal or gasket provides a fluid tight seal.

In some embodiments, the plunger diameter can be adjusted relative to a diameter of the cylinder-shaped barrel to adjust the overall fluid volume of the device. In one embodiment, the plunger further comprises a concave-shaped neck portion. In another embodiment, the concave-shaped neck portion forms an internal void space between the plunger and the side wall of the cylinder-shaped barrel. In one embodiment, the void has a volume of from about 15 μL to about 1500 μL.

In some embodiments, the second end of the plunger has a diameter smaller than a diameter of the cylinder-shaped barrel.

In some embodiments, the second end of the plunger further comprises one or more seals to create a fluid tight seal between the cylinder-wall and the plunger.

In some aspects, the present invention is directed to a method for collecting and dispensing a sample fluid, the method comprising: (a) providing a sample collection and dispensing device, the device comprising: (i) a plunger, the plunger having a linear axis, a first end, a second end and a first flow channel running along the linear axis of the plunger, wherein the first flow channel comprises a first end and a second end, wherein the first end comprises an open dispensing outlet and the second end is connected to a first exit hole in the side of the plunger; (ii) a sample collection sponge, wherein the sample collection sponge is attached to the second end of the plunger; and (iii) a cylinder-shaped barrel, wherein the cylinder-shaped barrel comprises a first open end for receiving the plunger and a second closed end, and wherein the cylinder-shaped barrel comprises a flexible side wall; and (b) collecting a sample fluid using the sponge; (c) inserting the plunger and sponge into the cylinder-shaped barrel; (d) depressing the plunger along the linear axis to compress the sponge against the closed end wall of the cylinder-shaped barrel and thereby forcing the sample fluid through the sponge and into the cylinder-shaped barrel; and (e) optionally storing the sample fluid in the sample collection and dispensing device.

In some embodiments, the sample collection sponge further operates as a filter. In one embodiment, the sample collection and dispensing device further comprises a filter, and wherein the filter is located between the sponge and the plunger.

In one embodiment, the method further comprises dispensing the sample fluid from the cylinder-shaped barrel by squeezing the flexible side walls of the cylinder-shaped barrel to force the sample fluid through the first flow channel and out of the open dispensing outlet.

In another embodiment, the sample collection device further comprises: (i) a concave-shaped neck portion in the plunger, the concave-shaped neck portion forming an internal space or void between the plunger and the side wall of the cylinder-shaped barrel; and (ii) a second flow channel connecting the second end of the plunger to a second exit hold in the side of the plunger; and wherein compression of the sponge against the closed end of the cylinder-shaped barrel in step (c) forces the sample fluid through the second fluid flow channel and into the void.

In one embodiment, the second exit hole comprises a T-shaped exit hole having two openings on opposite sides of the plunger.

In one embodiment, the internal void space has an internal volume of from about 10 μL to about 1500 μL. In another embodiment, the cylinder-shaped barrel has an internal volume of from about 20 μL to about 2000 μL.

In some embodiments, the sample collection device and storage device further comprises a removable cap, and wherein the removable cap is attached to the first end of the plunger to cap the dispensing outlet of the first flow channel.

In some embodiments, the plunger further comprises a circular-shaped plate at the dispensing end of the plunger and wherein the plate has a plate diameter, wherein the plate diameter is the same as, or larger than a diameter of the cylinder-shaped barrel, and wherein the plate is operable to cover the cylinder-shaped barrel.

In one embodiment, the plunger and the cylinder-shaped barrel further comprise threads, wherein the threads on the plunger are on the underside of the circular-shaped plate and the threads on the cylinder-shaped barrel are on the inner wall of the cylinder-shaped barrel, and wherein the threads of the plunder are capable of engaging the threads of the cylinder-shaped barrel. In one embodiment, the plate further comprises a seal or a gasket operable to close the open end of the cylinder-shaped barrel and optionally wherein the seal or gasket provides a fluid tight seal.

In some embodiments, the second end of the plunger has a diameter smaller than a diameter of the cylinder-shaped barrel and the second end of the plunger further comprises one or more seals to create a fluid tight seal between the cylinder-wall and the plunger.

In some embodiments, the sample fluid is saliva.

### BRIEF DESCRIPTION OF THE DRAWINGS

Having thus described the subject matter of the present invention in general terms, reference will now be made to the accompanying drawings, which are not necessarily drawn to scale, and wherein:

FIG. 1 and FIG. 2 illustrate perspective views of an example of the sample collection and dispensing device in a non-actuated state and in a “ready to actuate” or storage state, respectively;

FIG. 3 and FIG. 4 illustrate a block diagram and a perspective view, respectively, of an example of the sample collection and dispensing device in relation to a DMF device or cartridge;

FIG. 5 and FIG. 6 illustrate exploded perspective views of an example of the sample collection and dispensing device;

FIG. 7A, FIG. 7B, FIG. 8A, and FIG. 8B illustrate exploded side views of an example of the sample collection and dispensing device shown in FIG. 5 and FIG. 6;

FIG. 9A and FIG. 9B illustrate end views of an example of the sample collection and dispensing device shown in FIG. 5 and FIG. 6;

FIG. 10A, FIG. 10B, FIG. 11A, and FIG. 11B illustrate various cross-sectional views of an example of the sample collection and dispensing device shown in FIG. 5 and FIG. 6;

FIG. 12A and FIG. 12B illustrate a solid perspective view and a transparent perspective view, respectively, of an example of a plunger of the sample collection and dispensing device shown in FIG. 5 and FIG. 6;

in

FIG. 13A, FIG. 13B, FIG. 14A, and FIG. 14B illustrate various perspective views of an example of the plunger of the sample collection and dispensing device shown in FIG. 5 and FIG. 6;

FIG. 15A, FIG. 15B, FIG. 16A, and FIG. 16B illustrate various side views of an example of the plunger of the sample collection and dispensing device shown in FIG. 5 and FIG. 6;

FIG. 17A and FIG. 17B illustrate end views of an example of the plunger of the sample collection and dispensing device shown in FIG. 5 and FIG. 6;

FIG. 18A and FIG. 18B illustrate cross-sectional views of an example of the plunger of the sample collection and dispensing device shown in FIG. 5 and FIG. 6;

FIG. 19A and FIG. 19B illustrate a solid perspective view and a transparent perspective view, respectively, of an example of a barrel of the sample collection and dispensing device shown in FIG. 5 and FIG. 6;

FIG. 20A and FIG. 20B illustrate a perspective view and a side view, respectively, of an example of the barrel of the sample collection and dispensing device shown in FIG. 5 and FIG. 6;

FIG. 21A and FIG. 21B illustrate end views of an example of the barrel of the sample collection and dispensing device shown in FIG. 5 and FIG. 6;

FIG. 22A, FIG. 22B, and FIG. 22C illustrate a perspective view, a side view, and an end view, respectively, of an example of a sample collection sponge of the sample collection and dispensing device shown in FIG. 5 and FIG. 6;

FIG. 23A and FIG. 23B illustrate cross-sectional views of an example of the flow paths in and the operation of the sample collection and dispensing device shown in FIG. 5 and FIG. 6;

FIG. 24 illustrates a flow diagram of an example of a method of using the sample collection and dispensing device;

FIG. 25A, FIG. 25B, FIG. 25C, FIG. 25D, and FIG. 25E illustrate pictorially certain steps of the method of using the sample collection and dispensing device shown in FIG. 24;

FIG. 26 illustrates a perspective view of another example of the sample collection and dispensing device and shown in a “ready to actuate” state;

FIG. 27, FIG. 28, and FIG. 29 illustrate exploded perspective views of an example of the sample collection and dispensing device shown in FIG. 26;

FIG. 30A and FIG. 30B illustrate side views of an example of the sample collection and dispensing device shown in FIG. 26;

FIG. 31A and FIG. 31B illustrate end views of an example of the sample collection and dispensing device shown in FIG. 26;

FIG. 32A, FIG. 32B, FIG. 33A, and FIG. 33B illustrate various cross-sectional views of an example of the sample collection and dispensing device shown in FIG. 26;

FIG. 34A and FIG. 34B illustrate a solid perspective view and a transparent perspective view, respectively, of an example of a plunger of the sample collection and dispensing device shown in FIG. 26;

FIG. 35A, FIG. 35B, FIG. 36A, and FIG. 36B illustrate various perspective views of an example of the plunger of the sample collection and dispensing device shown in FIG. 26;

FIG. 37A, FIG. 37B, and FIG. 37C illustrate various side views of an example of the plunger of the sample collection and dispensing device shown in FIG. 26;

FIG. 38A and FIG. 38B illustrate end views of an example of the plunger of the sample collection and dispensing device shown in FIG. 26;

FIG. 39A and FIG. 39B illustrate cross-sectional views of an example of the plunger of the sample collection and dispensing device shown in FIG. 26;

FIG. 40A and FIG. 40B illustrate a solid perspective view and a transparent perspective view, respectively, of an example of a barrel of the sample collection and dispensing device shown in FIG. 26;

FIG. 41A and FIG. 41B illustrate a perspective view and a side view, respectively, of an example of the barrel of the sample collection and dispensing device shown in FIG. 26;

FIG. 42A and FIG. 42B illustrate end views of an example of the barrel of the sample collection and dispensing device shown in FIG. 26;

FIG. 43A, FIG. 43B, and FIG. 43C illustrate views of an example of certain process steps of using the sample collection and dispensing device shown in FIG. 26;

FIG. 44A and FIG. 44B illustrate cross-sectional views showing an example of the sample collection and dispensing device including a filter; and

FIG. 45A and FIG. 45B illustrate cross-sectional views of a sample collection and dispensing device in which the plunger and barrel engage via two sets of seal rings instead of threads.

### DETAILED DESCRIPTION

The subject matter now will be described more fully hereinafter with reference to the accompanying drawings, in which some, but not all embodiments of the subject matter are shown. Like numbers refer to like elements throughout. The subject matter may be embodied in many different forms and should not be construed as limited to the embodiments set forth herein; rather, these embodiments are provided so that this disclosure will satisfy applicable legal requirements. Indeed, many modifications and other embodiments of the subject matter set forth herein will come to mind to one skilled in the art to which the subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims.

In some embodiments, the subject matter provides sample collection and dispensing devices and methods.

In some embodiments, the sample collection and dispensing devices and methods may provide a plunger for holding a sample collection sponge, a fully or at least partly flexible and/or squeezable barrel for receiving the plunger and sample collection sponge, and a cap for capping a dispense outlet.

In some embodiments, the sample collection and dispensing devices and methods may provide a plunger capable of holding a sample collection sponge on one end and including a dispense outlet at its opposite end and wherein the plunger may include one or more flow channels for directing a precise amount to sample from the sample collection sponge to the dispense outlet.

In some embodiments, the sample collection and dispensing devices and methods may provide a plunger that includes a narrow neck portion for providing a compressible space or void when the plunger is installed in a flexible and/or squeezable barrel.

In some embodiments, the sample collection and dispensing devices and methods may provide method steps that may include, but are not limited to, collecting a sample volume using the plunger holding the sample collection sponge, inserting the plunger holding the sample collection sponge and sample volume fully into the flexible and/or squeezable barrel and thereby causing sample liquid to flow from the sponge in the barrel (because of compressing the sponge) into the one or more flow channels in the plunger, and then squeezing the flexible barrel at the neck portion of the plunger and thereby causing a substantially precise amount of sample liquid to flow from the one or more flow channels in the plunger and out of the dispense outlet because of the displacement action of squeezing the barrel.

In some embodiments, the sample collection and dispensing devices and methods may provide (1) an easy way to manually collect a sample (e.g., saliva), (2) a convenient way to store a sample (e.g., saliva), and (3) an easy way to manually dispense a precise amount of sample liquid (e.g., saliva) into, for example, a digital microfluidics (DMF) device or cartridge and/or into any standard lab equipment, such as a microwell plate (e.g., standard 96-well microplate), an Eppendorf tube, a polymerase chain reaction (PCR) tube, any standard lab sample holder/analyzer, and the like.

In some embodiments, the sample collection and dispensing devices and methods may provide a disposable single actuation device.

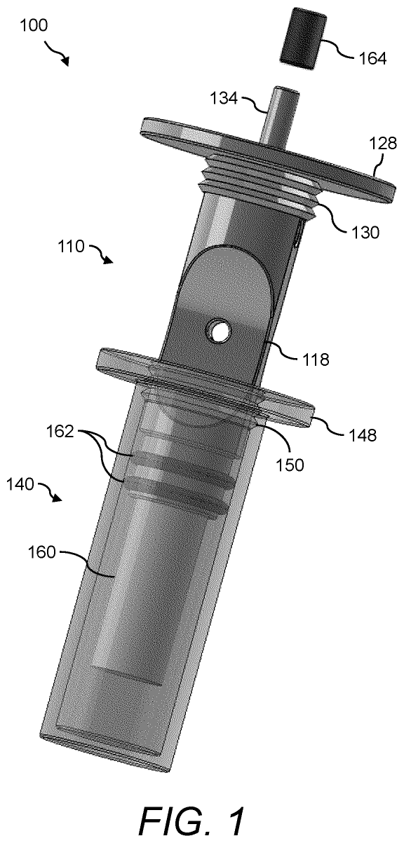

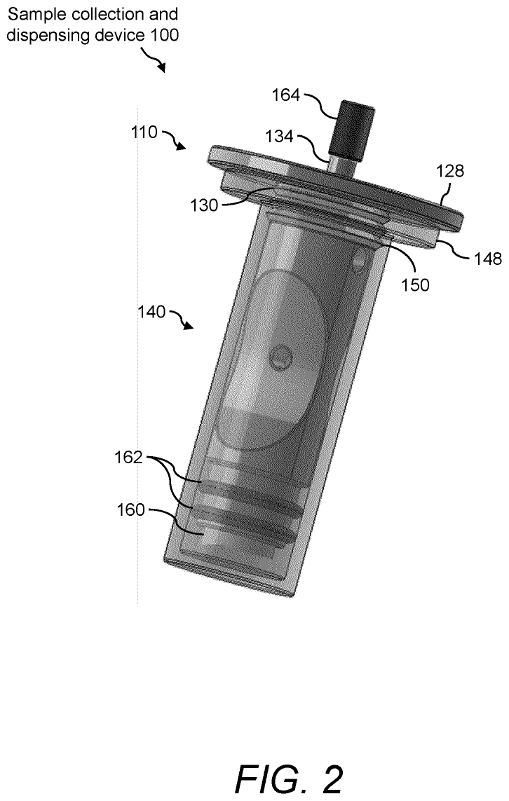

Referring now to FIG. 1 and FIG. 2 is perspective views of an example of the sample collection and dispensing device 100 in a non-actuated state and in a “ready to actuate” or storage state, respectively.

In this example, sample collection and dispensing device 100 may include a plunger 110 that is designed to be fitted into a barrel 140, a sample collection sponge 160, and a cap 164. Plunger 110 may be designed to hold sample collection sponge 160 on one end and to dispense liquid from the opposite end. The dispensing end of plunger 110 may include a plate 128 and a dispense outlet 134. Further, threads 130 may be provided around plunger 110 near the underside of plate 128. Further, a pair of seal rings 162 may be provided on the distal end of plunger 110.

Barrel 140 may be a cylinder-shaped barrel or reservoir that has an open end and a closed end. In one example, barrel 140 may be fully or at least partly flexible and/or squeezable.

In another example, barrel 140 may be include an element that may be fully or at least partly flexible and/or squeezable. In yet another example, barrel 140 may include an add-on component that may be fully or at least partly flexible and/or squeezable.

A rim 148 may be provide around the open end of barrel 140. Further, threads 150 may be provided inside the open end of barrel 140 and near rim 148. Threads 130 of plunger 110 are designed to engage with threads 150 of barrel 140 and thereby hold together plunger 110 and barrel 140 in sealed fashion.

Further, as plunger 110 and sample collection sponge 160 enter barrel 140, sample collection sponge 160 starts in an uncompressed state, as shown in FIG. 1. However, as plunger 110 and sample collection sponge 160 become fully engaged with barrel 140, sample collection sponge 160 becomes fully compressed, as shown in FIG. 2. Also, FIG. 2 shows dispense outlet 134 capped using cap 164. In this way, sample liquid may be stored within sample collection and dispensing device 100.

In sample collection and dispensing device 100, plunger 110 may include one or more flow channels for directing a precise amount to sample liquid from sample collection sponge 160 to dispense outlet 134. Further, plunger 110 may include a narrow neck portion for providing a compressible space or void when plunger 110 is installed within the flexible and/or squeezable barrel 140.

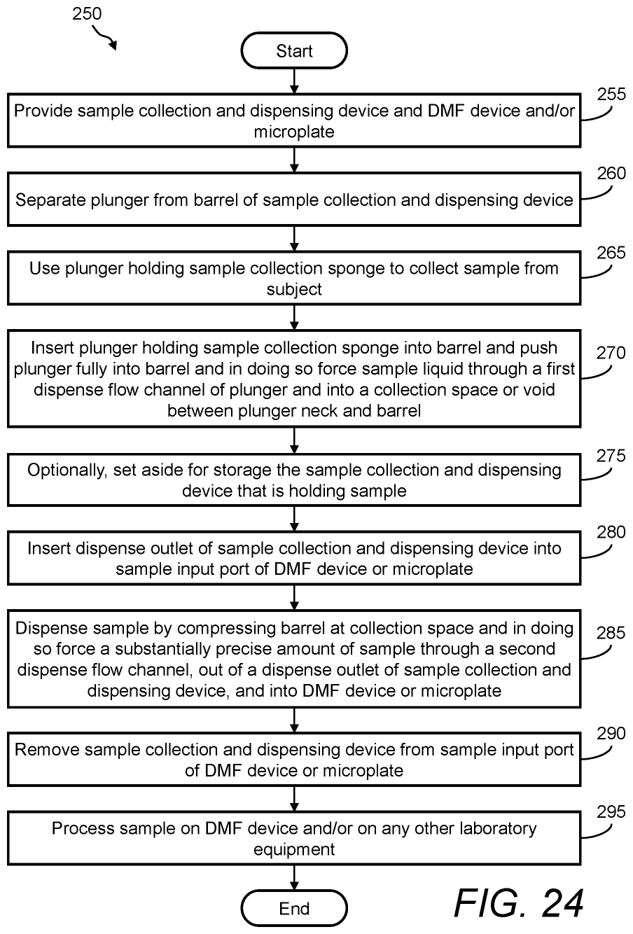

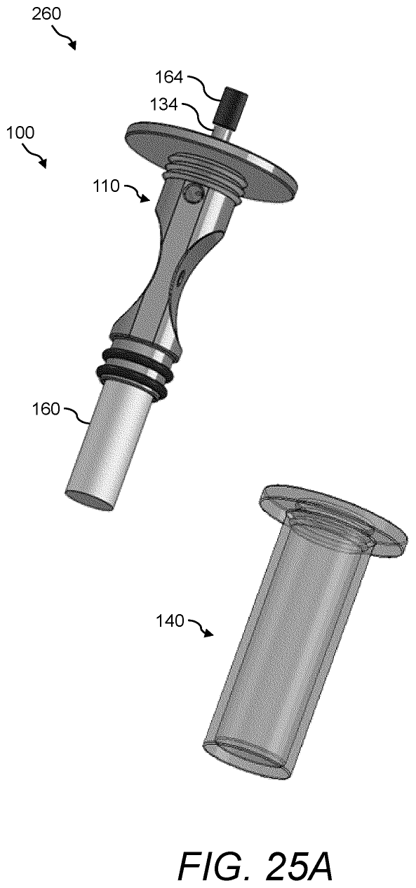

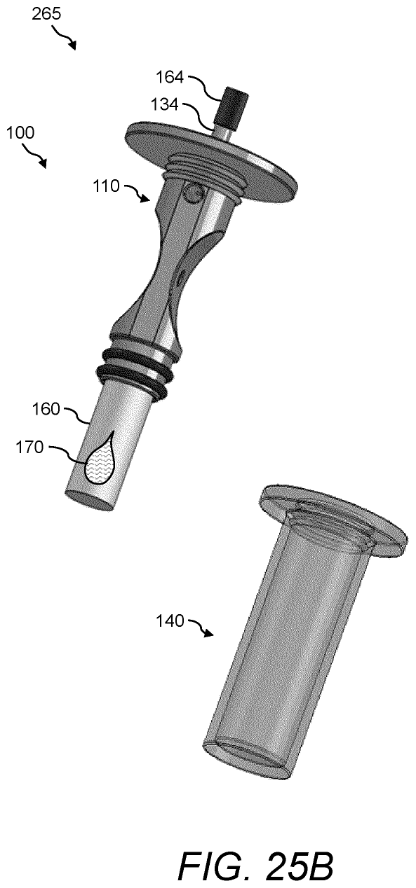

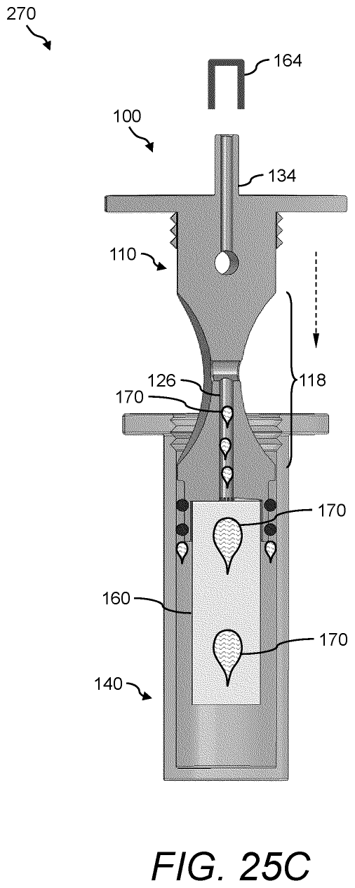

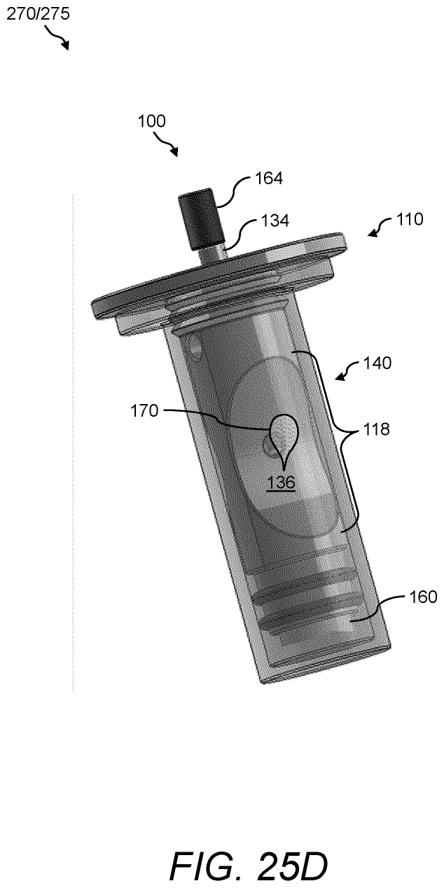

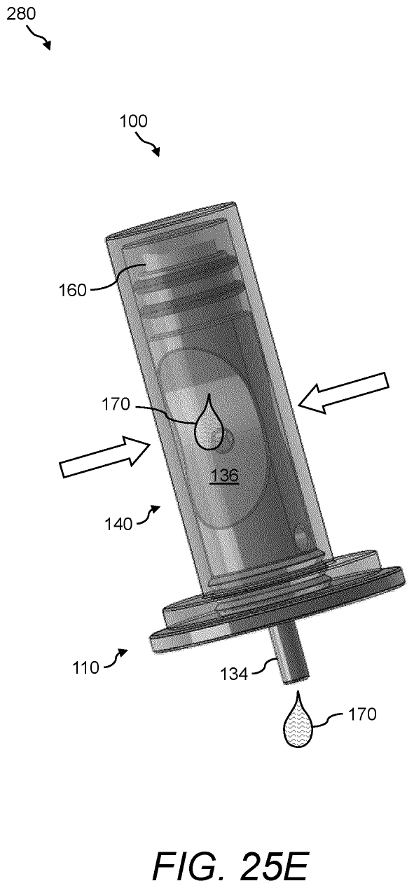

In some embodiments, a method of using sample collection and dispensing device 100 may include, but not are not limited to, the steps of collecting a sample volume using plunger 110 holding sample collection sponge 160, inserting plunger 110 holding sample collection sponge 160 and sample volume fully into the flexible and/or squeezable barrel 140 and thereby causing sample liquid to flow from sample collection sponge 160 in barrel 140 (because of compressing the sponge) into the one or more flow channels in plunger 110, and then squeezing the flexible barrel 140 at the neck portion of plunger 110 and thereby causing a substantially precise amount of sample liquid to flow from the one or more flow channels in plunger 110 and out of dispense outlet 134 because of the displacement action of squeezing barrel 140. More details of an example of method of using sample collection and dispensing device 100 are shown and described hereinbelow with reference to FIG. 24 through FIG. 25D.

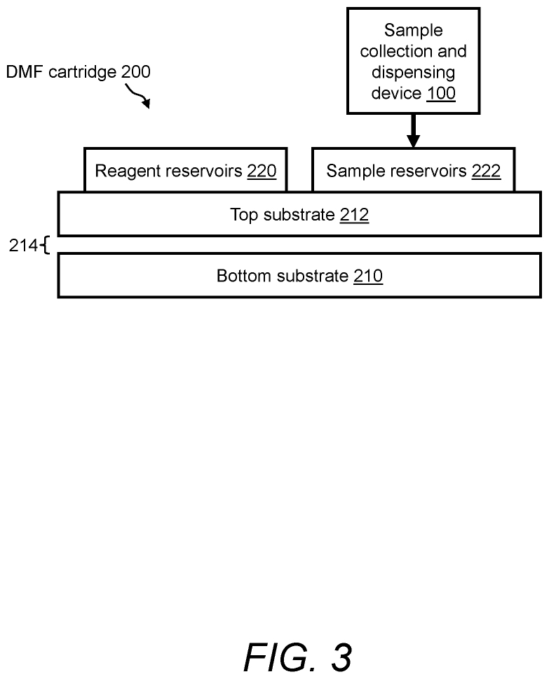

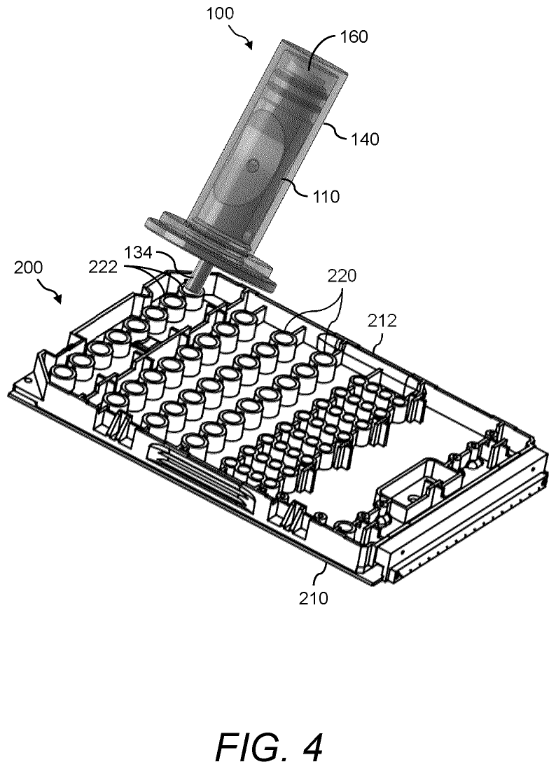

In some embodiments, the sample collection and dispensing device 100 may provide (1) an easy way to manually collect a sample (e.g., saliva), (2) a convenient way to store a sample (e.g., saliva), and (3) an easy way to manually dispense a precise amount of sample liquid (e.g., saliva) into, for example, a digital microfluidics (DMF) device or cartridge and/or a microwell plate, such as a standard 96-well microplate, as shown in FIG. 3 and FIG. 4.

Referring now to FIG. 3 and FIG. 4 is a block diagram and a perspective view, respectively, of an example of the sample collection and dispensing device 100 in relation to a DMF device or cartridge. For example, FIG. 3 and FIG. 4 show sample collection and dispensing device 100 in relation to a DMF device (or cartridge) 200. Sample collection and dispensing device 100 is not limited to use with a DMF device or cartridge only. Sample collection and dispensing device 100 may be used in like manner to dispense into any standard lab equipment, such as a microwell plate (e.g., standard 96-well microplate), an Eppendorf tube, a PCR tube, any standard lab sample holder/analyzer, and the like.

DMF device 200 may include, for example, a bottom substrate 210 and a top substrate 212 separated by a droplet operations gap 214 that may form a chamber in which droplet operations may be performed. Bottom substrate 210 may be, for example, a silicon or printed circuit board (PCB) substrate (see FIG. 4). Top substrate 212 may be, for example, a glass or plastic substrate (see FIG. 4). DMF device 200 may facilitate DMF capabilities generally for fluidic actuation including droplet transporting, merging, splitting, dispensing, diluting, and the like. One application of these DMF capabilities may be sample preparation. DMF device 200 may be provided, for example, as a disposable and/or reusable cartridge.

In one example, DMF device 200 may include any number and/or arrangements of reagent reservoirs 220 and sample reservoirs 222. More specifically, FIG. 4 shows dispense outlet 134 of sample collection and dispensing device 100 in relation to the loading port of the sample reservoirs 222. That is, dispense outlet 134 of sample collection and dispensing device 100 may be fitted into the loading port of a sample reservoir 222 and then sample liquid may be dispensed from sample collection and dispensing device 100 into DMF device 200.

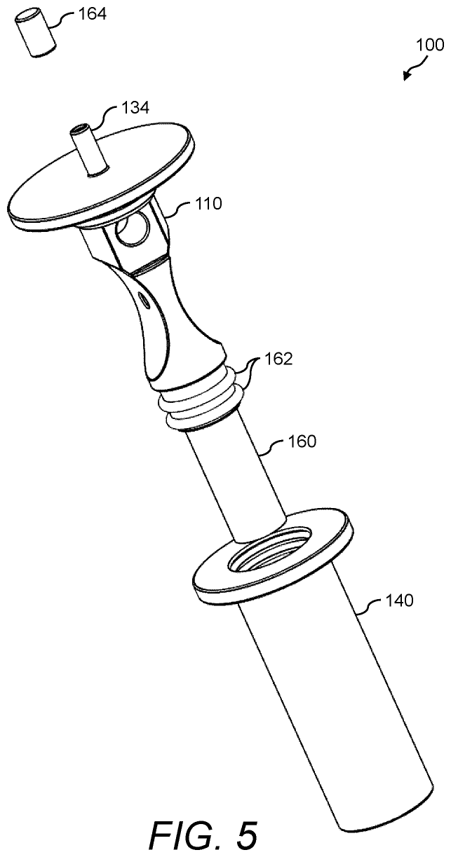

More details of an example of sample collection and dispensing device 100 are shown and described hereinbelow with reference to FIG. 5 through FIG. 11B. For example, FIG. 5 and

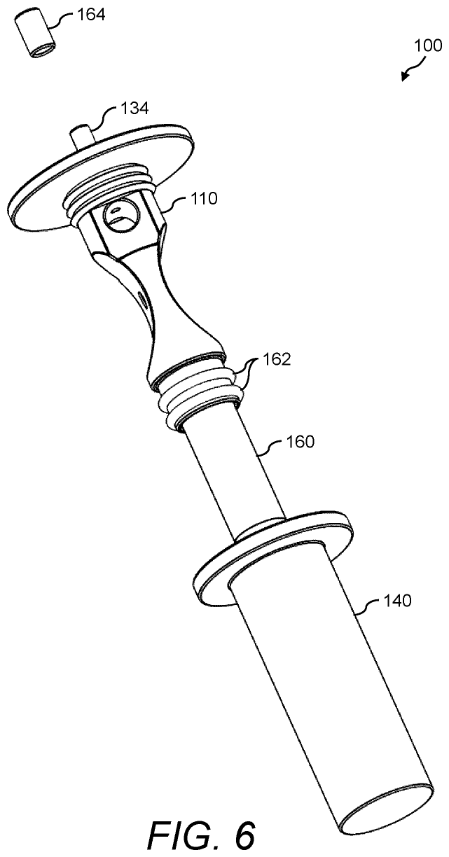

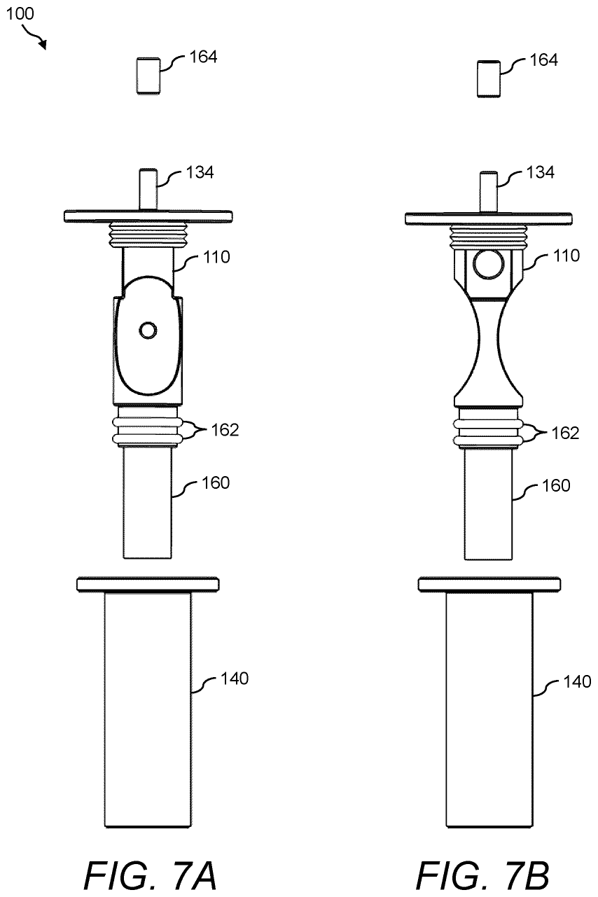

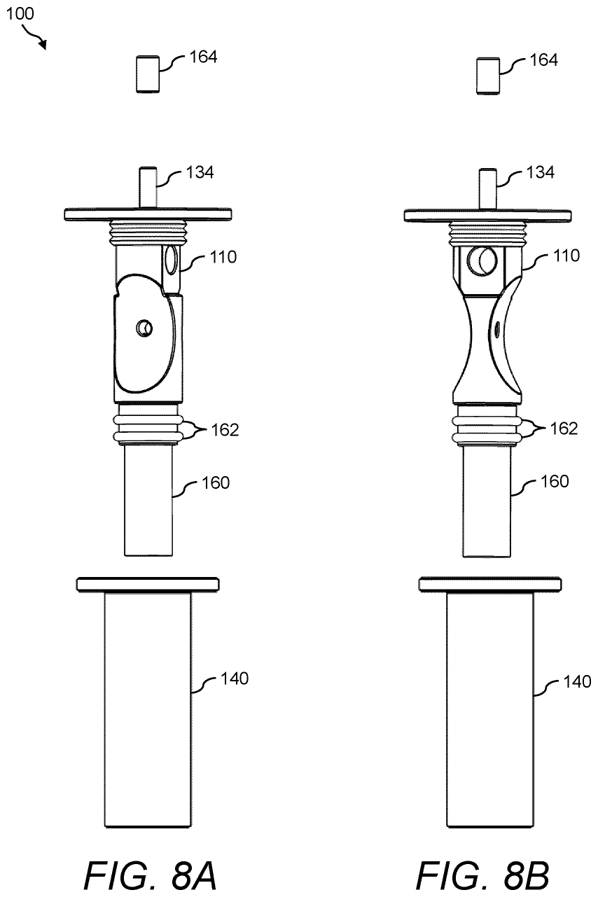

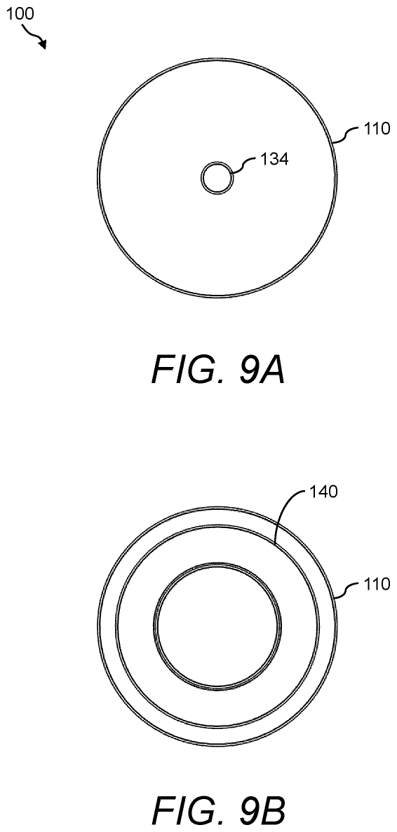

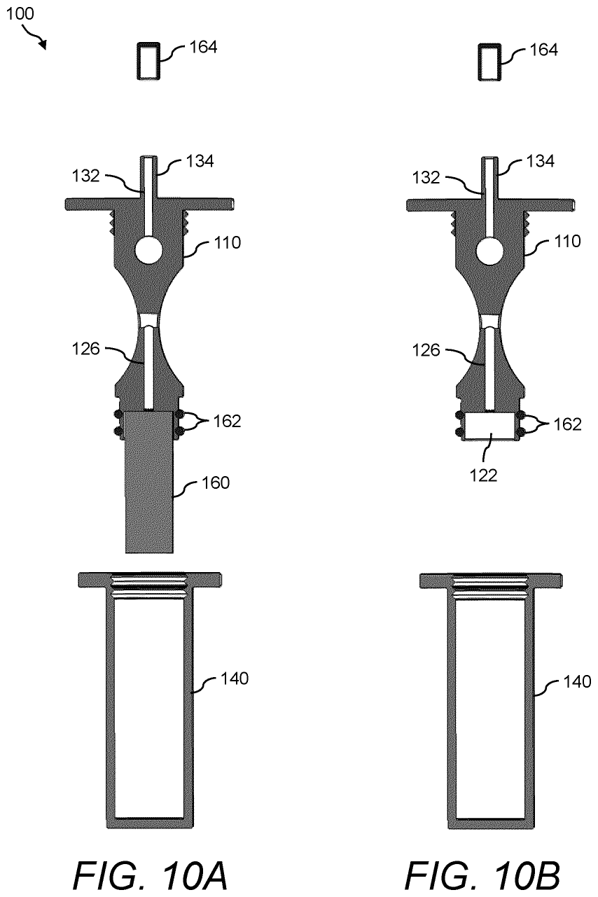

FIG. 6 show exploded perspective views of sample collection and dispensing device 100. FIG. 7A, FIG. 7B, FIG. 8A, and FIG. 8B show exploded side views of an example of sample collection and dispensing device 100. FIG. 9A and FIG. 9B show end views of sample collection and dispensing device 100. FIG. 10A, FIG. 10B, FIG. 11A, and FIG. 11B show various cross-sectional views of sample collection and dispensing device 100.

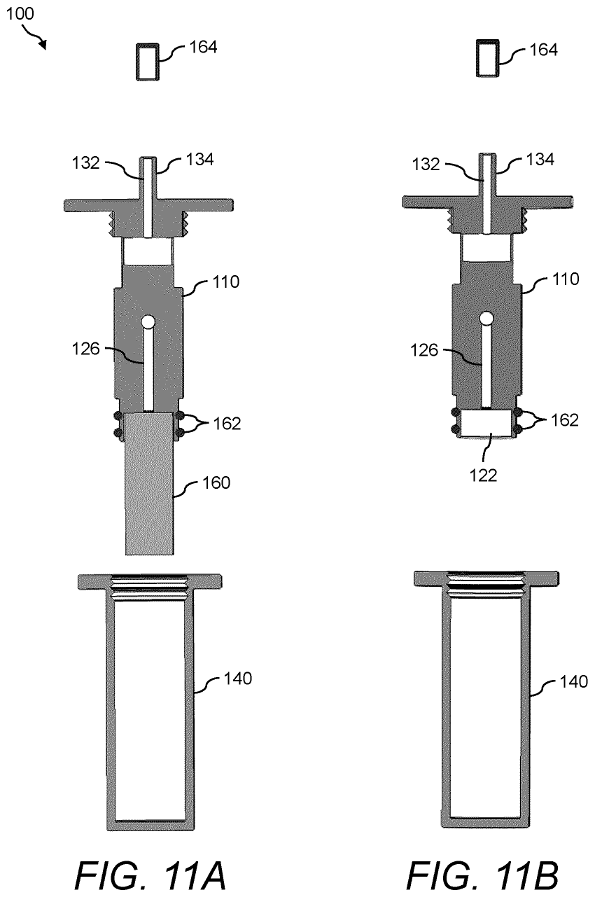

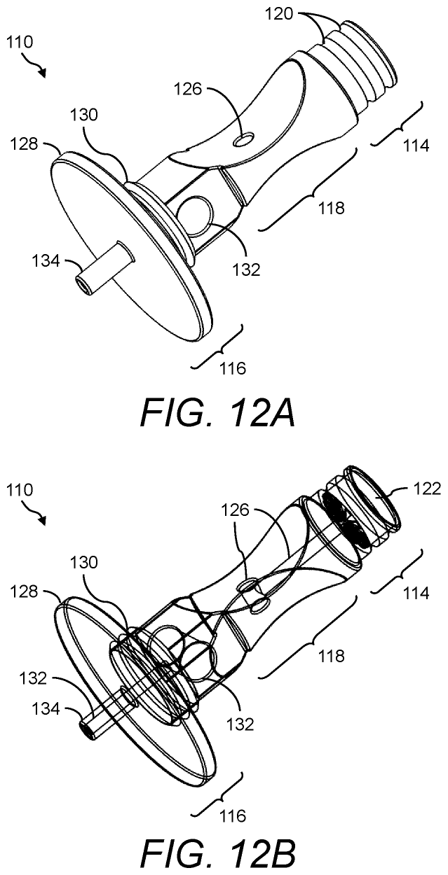

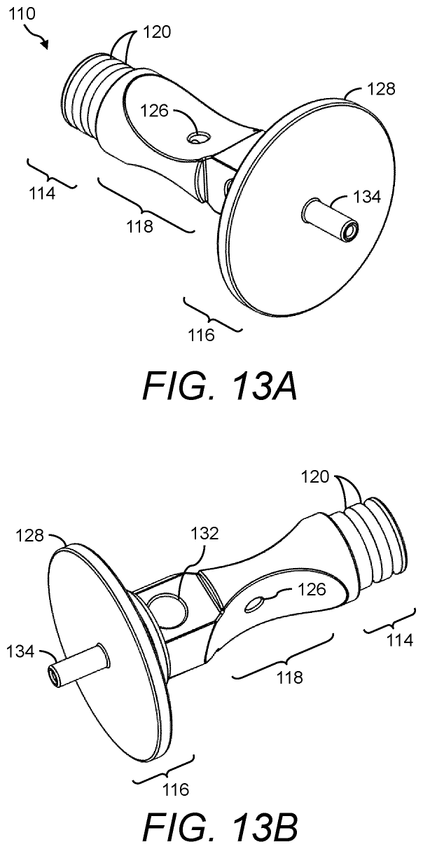

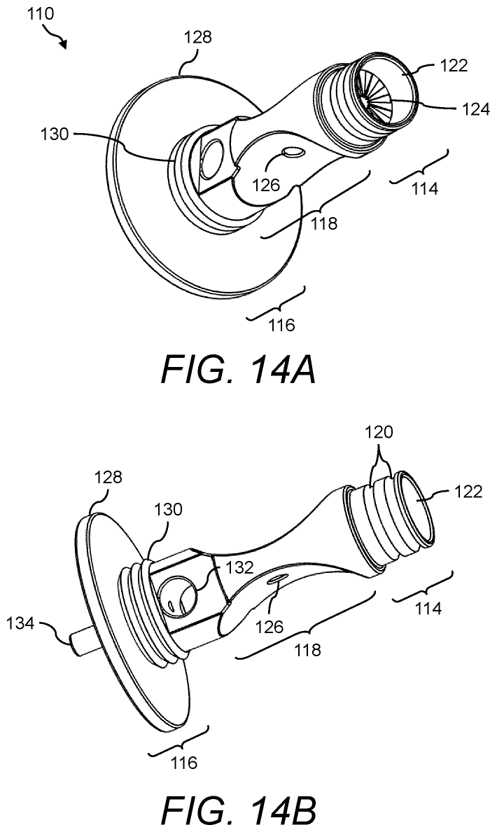

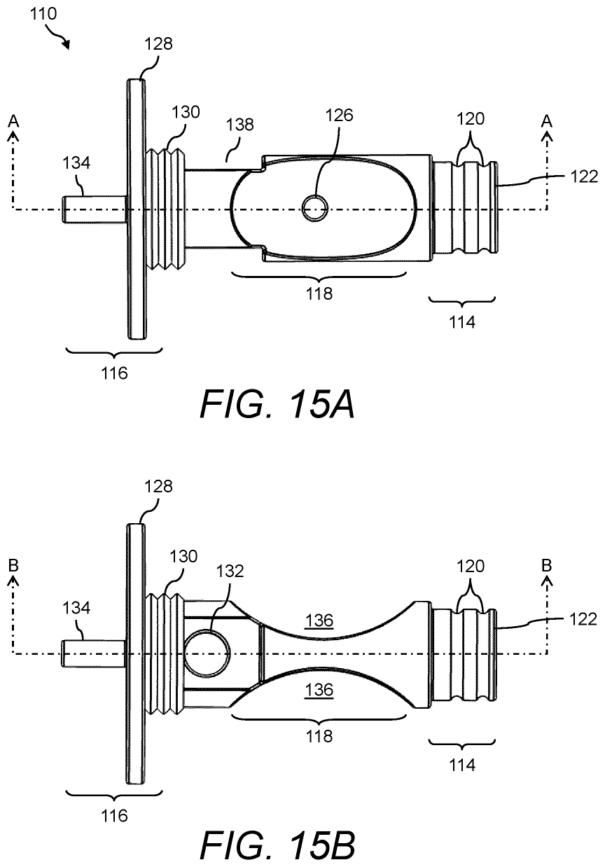

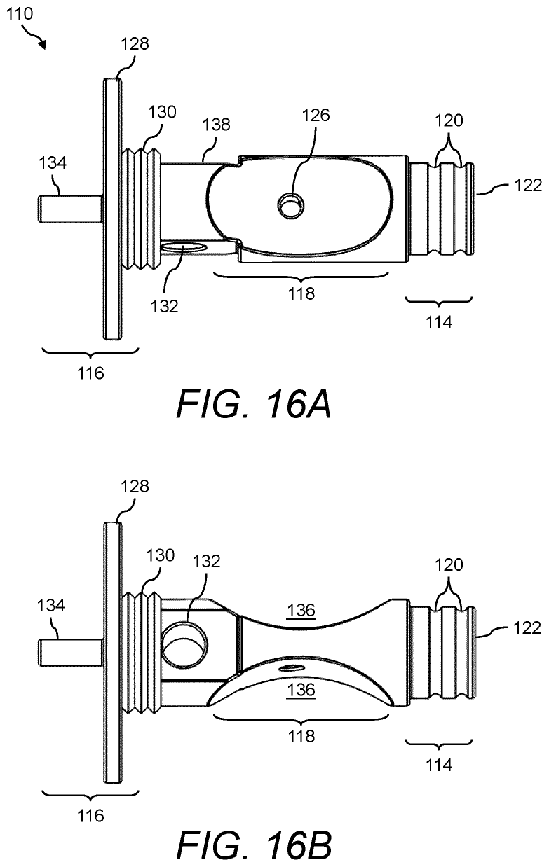

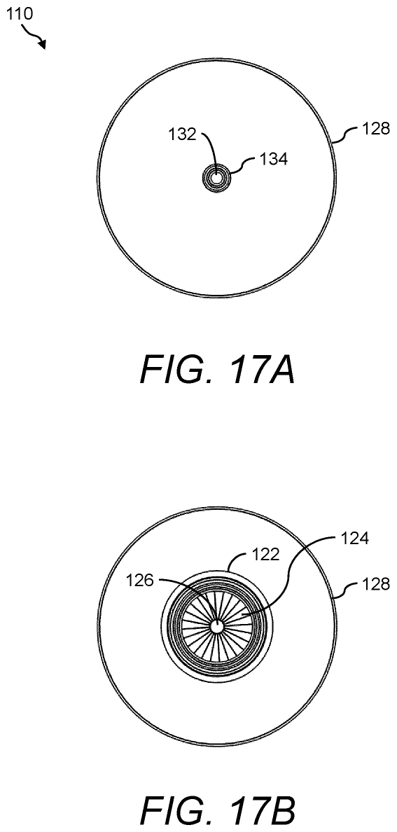

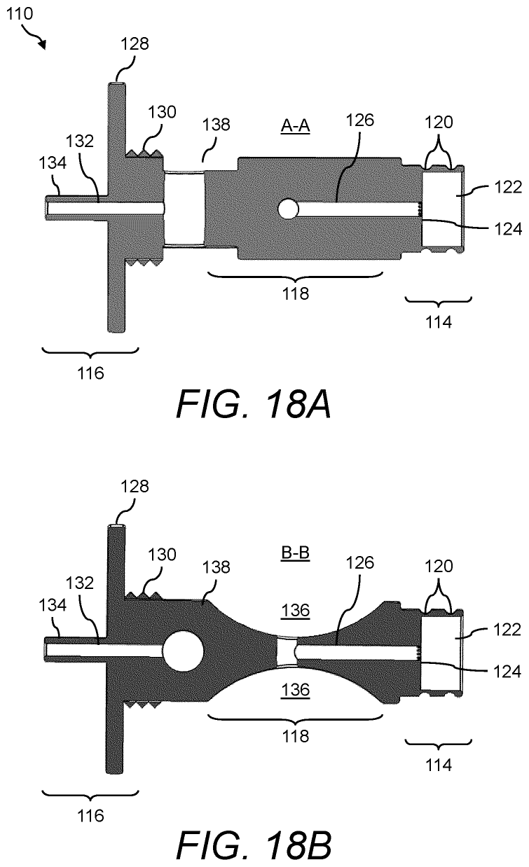

More details of an example of plunger 110 of the sample collection and dispensing device 100 are shown and described hereinbelow with reference to FIG. 12A through FIG. 18B. For example, FIG. 12A and FIG. 12B show a solid perspective view and a transparent perspective view, respectively, of plunger 110. FIG. 13A, FIG. 13B, FIG. 14A, and FIG. 14B show various perspective views of plunger 110. FIG. 15A, FIG. 15B, FIG. 16A, and FIG. 16B show various side views of plunger 110. FIG. 17A and FIG. 17B show end views of plunger 110. FIG. 18A and FIG. 18B show cross-sectional views of plunger 110.

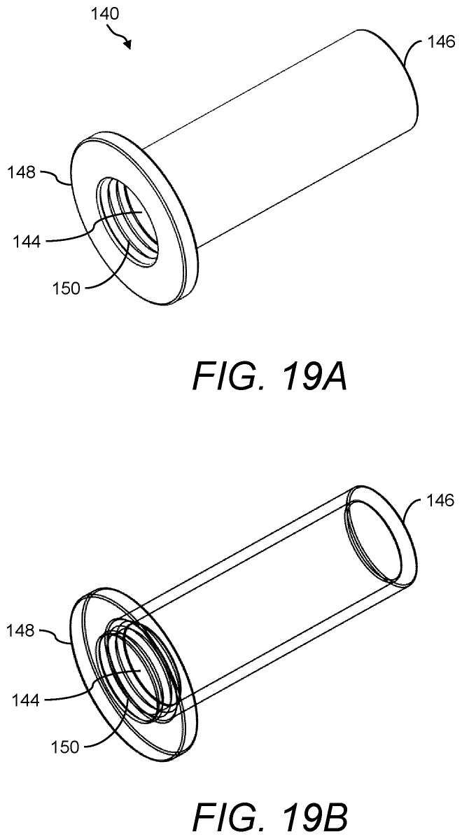

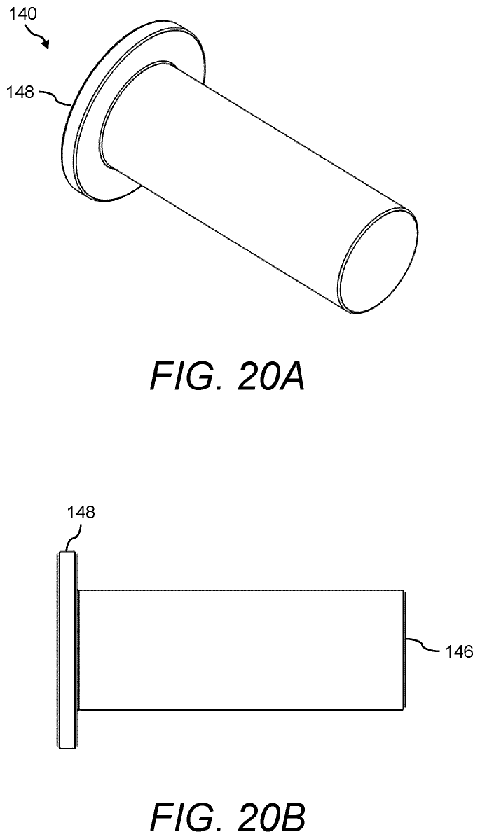

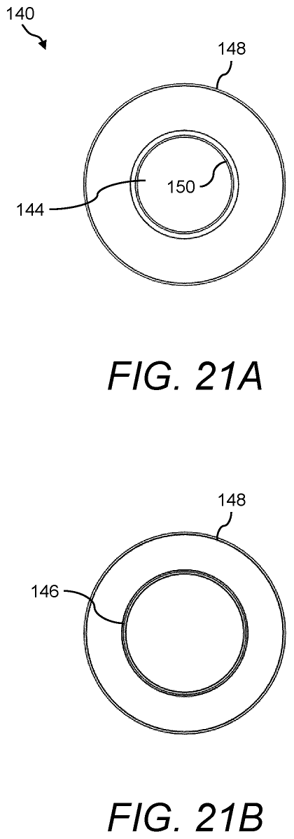

More details of an example of barrel 140 of the sample collection and dispensing device 100 are shown and described hereinbelow with reference to FIG. 19A through FIG. 21B. For example, FIG. 19A and FIG. 19B show a solid perspective view and a transparent perspective view, respectively, of barrel 140. FIG. 20A and FIG. 20B show a perspective view and a side view, respectively, of barrel 140. FIG. 21A and FIG. 21B show an open end view and a closed end view, respectively, of barrel 140.

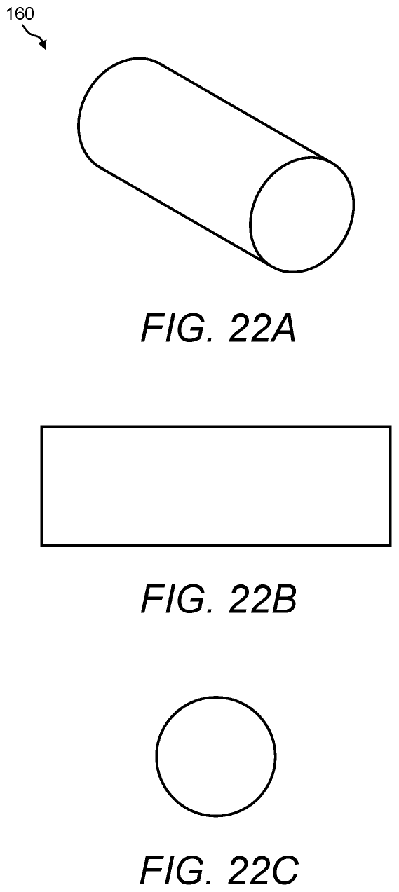

More details of an example of sample collection sponge 160 of the sample collection and dispensing device 100 are shown and described hereinbelow with reference to FIG. 22A through FIG. 22C. For example, FIG. 22A, FIG. 22B, and FIG. 22C is a perspective view, a side view, and an end view, respectively, of sample collection sponge 160.

Referring now again to FIG. 5 through FIG. 22C, sample collection and dispensing device 100 may include plunger 110, barrel 140, sample collection sponge 160, and cap 164.

Plunger 110 has a collection end 114 and a dispensing end 116. Further, a central region of plunger 110 may include a concave-shaped neck portion 118 (see FIG. 12A through FIG. 18B). A pair of seal ring grooves 120 may be provided around collection end 114 of plunger 110 for holding a corresponding pair of seal rings 162. Seal rings 162 may be, for example, rubber O-rings or gaskets. A sponge receptacle 122 may be provided at collection end 114 of plunger 110 for holding sample collection sponge 160. Sponge receptacle 122 may include a textured floor 124 (see FIG. 14A). Sponge receptacle 122 supplies one end of a first stage flow channel 126, which may be, for example, a T-channel. Further, the T-portion of flow channel 126 opens to the sides of a neck portion 118 of plunger 110. Accordingly, the T-portion of flow channel 126 supplies a space or void 136 around the neck portion 118 of plunger 110 (see FIG. 15B, FIG. 16B, FIG. 18B). In one example, flow channel 126 may be sized to hold some desired amount of liquid anywhere in the range of from about 10 μL to about 1500 μL.

A plate 128 may be arranged at dispensing end 116 of plunger 110. Threads 130 may be provided at dispensing end 116 of plunger 110 and near the underside of plate 128. A second stage flow channel 132, which may be, for example, a T-channel may be provided at dispensing end 116 of plunger 110. For example, a T-portion of flow channel 132 is oriented toward neck portion 118 of plunger 110 and opens into a space or void 138 (see FIG. 15A, FIG. 16A, FIG. 18A). Then, the other end of flow channel 132 extends through plate 128 and supplies dispense outlet 134 of plunger 110. Further, when plunger 110 is inside barrel 140 there may be two spaces or voids 136 at the concave-shaped neck portion 118 of plunger 110. Together, the two flow channels 132, the two flow channels 136, and the two spaces or voids 136 may be sized to hold and dispense some desired amount of liquid anywhere in the range of from about 10 μL to about 1500 μL in one example, from about 100 μL to about 200 μL in another example, or about 150 μL in yet another example.

In one example, plunger 110 may be formed of thermoplastic material using an injection molding process. Accordingly, plunger 110 may be substantially rigid and lightweight. In one example, the overall length of plunger 110 may be from about 10 mm to about 100 mm. In one example, the 10 mm-plunger 110 may be used to process small-volume samples. In another example, the 100 mm-plunger 110 may be used to process large-volume samples. Further, plunger 110 may be from about 20 mm to about 50 mm in diameter. Further, the depth of sponge receptacle 122 may be from about 2 mm to about 20 mm in one example, or from about 0.5 mm to about 2 mm in another example. Further, plate 128 may be from about 0.5 mm to about 5 mm thick and from about 20 mm to about 50 mm in diameter in one example, or from about 50 mm to about 100 mm in diameter in another example. Further, to mate to, for example, standard DMF devices and/or microplates, dispense outlet 134 may be from about 3 mm to about 30 mm long and from about 1 mm to about 10 mm in diameter. Further, cap 164 may be sized to snap-fit onto dispense outlet 134.

Barrel 140 may be the cylinder-shaped barrel or reservoir that has an open end 144 and a closed end 146. Again, rim 148 may be around the outside of open end 144 of barrel 140 and threads 150 may be around the inside of open end 144 of barrel 140 near rim 148.

In one example, barrel 140 may be formed of thermoplastic material using an injection molding process. In one example, the walls of the cylinder-shaped barrel 140 may be formed suitably thin to provide flexibility thereof. For example, the walls of barrel 140 may be formed suitably thin to allow the sides of barrel 140 to be squeezed or compressed inward (see FIG. 25D). The dimensions of barrel 140 may vary depending on the size of plunger 110.

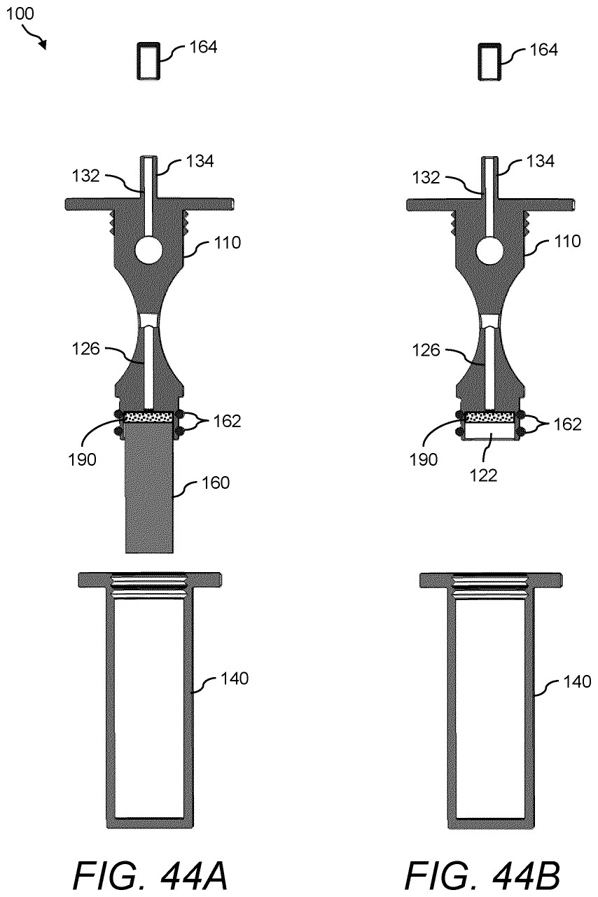

Further, sample collection sponge 160 may be a standard collection sponge or bio-sponge. Further, in sample collection and dispensing device 100, sample collection sponge 160 may be a sample collection mechanism only or both a sample collection mechanism and filter mechanism. In other embodiments, dispensing device 100 may include both a sample collection sponge 160 and a filter, as shown, for example, in FIG. 44.

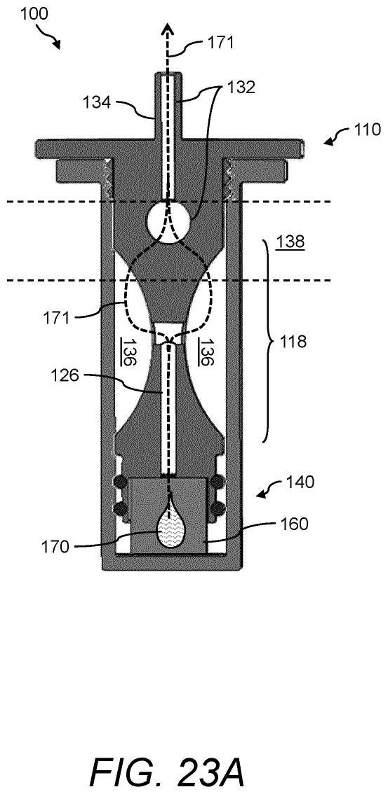

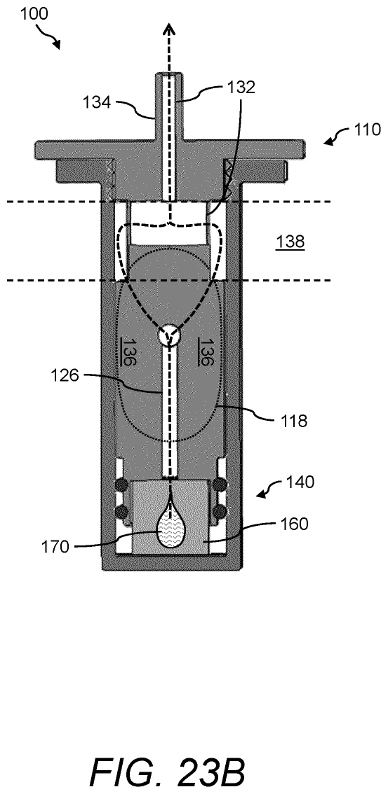

Referring now to FIG. 23A and FIG. 23B is cross-sectional views of an example of the flow paths in and the operation of the sample collection and dispensing device 100 shown in FIG. 5 and FIG. 6. For example, FIG. 23A and FIG. 23B shows that when sample collection sponge 160 holding some volume of sample (e.g., sample 170) is compressed within barrel 140, a sample flow 171 occurs from sample collection sponge 160 through the first dispense flow channel 126 of plunger 110 and then into a collection space or void 136 between neck portion 118 of plunger 110 and the walls of barrel 140. Next, when the sides of barrel 140 are squeezed or compressed together near neck portion 118 of plunger 110, the sample flow 171 continues into the space or void 138 near the threads 130 of plunger 110, and then enters the T-portion of the second dispense flow channel 132, then out of dispense outlet 134 of sample collection and dispensing device 100.

Generally, the operation of sample collection and dispensing device 100 may be as follows.
(1) Collect the sample (e.g., sample 170) using plunger 110 and collection sponge 160.
(2) Using plunger 110 and collection sponge 160, the sample (e.g., sample 170) may be pressed into the empty volume of barrel 140 and wherein the available volume of this space may be the empty volume of barrel 140 minus the volume of plunger 110.
(3) Maintain sample collection and dispensing device 100 with the dispense outlet 134-end oriented upward until ready for use (i.e., dispensing).
(4) The collection space or void 136 between plunger 110 and the squeezable part of barrel 140 form a precise volume, which is a part of the available volume in sample collection and dispensing device 100.
(5) By squeezing barrel 140, this precise volume may be displaced out of sample collection and dispensing device 100.

Accordingly, the operation of sample collection and dispensing device 100 may depend on two steps or actions: (1) the sample collection sponge 160 holding some volume of sample (e.g., sample 170) being compressed within barrel 140, and (2) the sides of barrel 140 being squeezed or compressed together near neck portion 118 of plunger 110. The volume of sample 170 dispensed substantially correlates to the volume of the collection space or void 136 between neck portion 118 of plunger 110 and barrel 140. Accordingly, the dispense precision of sample collection and dispensing device 100 may be provided by setting the volume of the collection space or void 136.

Referring now again to FIG. 1 through FIG. 23B, other features of the presently disclosed sample collection and dispensing device 100 are possible. For example, sample collection and dispensing device 100 described hereinabove may be called “2-sided” because neck portion 118 of plunger 110 provides two collection spaces or voids 136 (i.e., one on each side) that may be squeezed or compressed to dispense liquid. However, in other embodiments, sample collection and dispensing device 100 may be called “1-sided” because neck portion 118 of plunger 110 may include one collection space or void 136 only (i.e., on one side only) that may be squeezed or compressed to dispense liquid.

Further, in other embodiments, the walls of barrel 140 of sample collection and dispensing device 100 may be designed to automatically snap into collection spaces or voids 136 when squeezed. That is, the walls of barrel 140 may snap like a spring into collection spaces or voids 136 when squeezed and then remain collapsed inward when the squeeze is released. For example, the walls of barrel 140 may snap itself completely into collection spaces or voids 136 like a spring when a point of no return has been reached. In this example, the walls of barrel 140 may be formed of non-elastic material.

Further, in other embodiments, instead of squeezing by hand, a separate mechanism or object may be used to squeeze the walls of barrel 140. In one example, a ring may be provided that can be pushed onto barrel 140. Then, this dispensing ring may be used to press into the collection spaces or voids 136 and induce the force on the barrel wall like a camshaft (e.g., the ring may have a camshaft-like bump inside).

Further, in the presently disclosed sample collection and dispensing device 100 it may be important to avoid a second dispense operation. This is because a second push may eventually lead to more fluid being dispensed than is desired or expected. Certain features, such as the spring-like walls of barrel 140 and/or the dispensing ring, may serve to ensure a single dispense operation only by preventing a second dispense operation.

Further, in the presently disclosed sample collection and dispensing device 100 it may be important to prevent the pull-back of air and/or sample back into the device. Again, certain features, such as the spring-like walls of barrel 140 and/or the dispensing ring, may serve to prevent the pull-back of air and/or sample back into sample collection and dispensing device 100.

Referring now to FIG. 24 is a flow diagram of an example of a method 250 of using the sample collection and dispensing device 100. Additionally, FIG. 25A, FIG. 25B, FIG. 25C,

FIG. 25D, and FIG. 25E show pictorially certain steps of method 250 shown in FIG. 24. Method 250 may include, but is not limited to, the following steps.

At a step 255, a sample collection and dispensing device and a DMF device and/or microplate are provided. For example, the sample collection and dispensing device 100 as described hereinabove with reference to FIG. 1 through FIG. 22C may be provided. Additionally, a DMF device (e.g., DMF device 200 shown in FIG. 4) and/or a microplate (e.g., 96-well microplate) may be provided.

At a step 260, the plunger and barrel of the sample collection and dispensing device are separated. For example and referring now to FIG. 25A, the user separates plunger 110 from the barrel 140 of sample collection and dispensing device 100. Then, the user may set aside the barrel 140. In one example, sample collection sponge 160 may come pre-installed (and non-removeable on plunger 110.

In another example, sample collection sponge 160 and sample collection and dispensing device 100 may be provided separately. Then, the user collects the sample with sample collection sponge 160. Then, the user may drop sample collection sponge 160 into barrel 140. Then, the user may insert plunger 110 into barrel 140 to squeeze out the sample collection sponge 160 into collection spaces or voids 136 of plunger 110.

In yet another example, sample collection sponge 160 and sample collection and dispensing device 100 may be provided separately. Then, the user may install by hand one end of sample collection sponge 160 into sponge receptacle 122 of plunger 110.

At a step 265, the plunger holding the sample collection sponge is used to collect a sample from a subject. For example and referring now to FIG. 25B, the user may use plunger 110 that is holding sample collection sponge 160 to collect a sample 170 (e.g., saliva) from the mouth of the subject.

At a step 270, the plunger holding the sample collection sponge is inserted into the barrel of the sample collection and dispensing device. Then, the plunger and sponge are pushed fully into the barrel. In doing so, sample liquid is forced through a first dispense flow channel of the plunger and into a collection space or void between the plunger neck and the barrel.

For example and referring now to FIG. 25C and FIG. 25D, the user may remove cap 164 so that air can vent out of sample collection and dispensing device 100. Then, the user may insert plunger 110 holding sample collection sponge 160 into barrel 140 of sample collection and dispensing device 100. Then, the user pushes plunger 110 and sample collection sponge 160 fully into barrel 140. Then, the user screws plunger 110 and barrel 140 together in sealed fashion via threads 130 of plunger 110 and threads 150 of barrel 140. However, in other embodiments (not shown), instead of coupling plunger 110 and barrel 140 together using a screw fit with threads, other types of connections may be provided, such as a friction lock or clip, a snap-fit, a plug-in connection, and the like.

In doing so, sample collection sponge 160 is compressed and causing sample 170 (e.g., saliva) to be forced through the first dispense flow channel 126 of plunger 110 and into a collection space or void 136 between neck portion 118 of plunger 110 and the walls of barrel 140. Further, there is a collection space or void 136 on each side of neck portion 118. The two spaces or voids 136 between neck portion 118 of plunger 110 and barrel 140 may have a certain substantially precise volume that may be set by the shape and/or size of neck portion 118 of plunger 110. The total volume of the two flow channels 132, the two flow channels 136, and the two spaces or voids 136 may be designed to hold any desired amount in the range of from about 10 μL to about 1500 μL in one example, from about 100 μL to about 200 μL in another example, or may be about 150 μL in yet another example.

In this step, sample 170 (e.g., saliva) may not necessarily be collected/stored in collection spaces or voids 136. Albeit, these collection spaces or voids 136 create a precise volume that can be displaced by pressing barrel 140. For example, sample 170 may already be present further downstream toward dispense outlet 134.

Optionally in step 270, to assist the flow of sample liquid, a few drops of other liquid, such as reagent, saline, buffer, or surfactants (e.g., Tween 20, Tetronic 90R4, Pluronic), may be added into barrel 140 prior to inserting sample collection sponge 160 holding the sample 170 into barrel 140. In other embodiments, sample collection sponge 160 may be preloaded with some volume of, for example, buffer solution. In other embodiments, sample collection sponge 160 may be provided with a few drops of liquid dried within the sponge or within a filter or insert that comes after the sponge. In other embodiments, a blister pack holding some liquid may be provided inside barrel 140 and wherein the blister pack ruptures when plunger 110 and sample collection sponge 160 are pushed into barrel 140.

At an optional step 275, the sample collection and dispensing device holding the sample is set aside for storage. For example and referring now to FIG. 25D, the user may install cap 164 onto dispense outlet 134. Then, the user may set aside for storage the sample collection and dispensing device 100 that is holding sample 170 (e.g., saliva) and capped with cap 164.

At a step 280, a dispense outlet of the sample collection and dispensing device is inserted into a sample input port of a DMF device and/or microplate. For example and referring now to FIG. 4, the user may remove cap 164 from dispense outlet 134 of sample collection and dispensing device 100. Then, for example, the user may insert dispense outlet 134 of sample collection and dispensing device 100 into a sample input port of a DMF device (e.g., DMF device 200 shown in FIG. 4) or a microplate (e.g., 96-well microplate).

At a step 285, the sample is dispensed by compressing the barrel at the collection space of the sample collection and dispensing device. In doing so, a substantially precise amount of sample liquid is forced through a second dispense flow channel, out of the dispense outlet of sample collection and dispensing device, and into the DMF device or a microplate.

For example and referring now to FIG. 25E, the user may use his/her fingers to squeeze or compress the sides of barrel 140 of sample collection and dispensing device 100. More specifically, the user may use his/her fingers to squeeze or compress barrel 140 at the two spaces or voids 136 at the sides of neck portion 118 of plunger 110. In some embodiments there may be only one collection space or void 136 on one side of neck portion 118 of plunger 110, instead of two collection spaces or voids 136 (one on each side). In this way, the walls of barrel 140 collapse against the sides of neck portion 118 of plunger 110, which displaces any sample 170 in this collection space and forces the sample 170 into space or void 138, the entering the T-portion of the second dispense flow channel 132, then out of dispense outlet 134 of sample collection and dispensing device 100, and then into the DMF device (e.g., DMF device 200 shown in FIG. 4) or microplate (e.g., 96-well microplate). Accordingly, sample 170 may be dispensed using the sample collection and dispensing device 100. Sample collection and dispensing device 100 may be sized to dispense some desired amount of sample 170 (e.g., saliva) anywhere in the range of from about 10 μL to about 1500 μL in one example, from about 100 μL to about 200 μL in another example, or about 150 μL in yet another example.

At a step 290, the sample collection and dispensing device is removed from the sample input port of the DMF device or microplate. For example, the user may disengage dispense outlet 134 of sample collection and dispensing device 100 from the sample input port of the DMF device (e.g., DMF device 200 shown in FIG. 4) or microplate (e.g., 96-well microplate). Optionally, sample collection and dispensing device 100 may be discarded.

At a step 295, the sample is processed on a DMF device and/or on any other laboratory equipment. For example, using any means of processing biological materials, the sample 170 (e.g., saliva) may be processed on a DMF device (e.g., DMF device 200 shown in FIG. 4) and/or on any standard lab equipment, such as a microwell plate (e.g., standard 96-well microplate), an Eppendorf tube, a PCR tube, any standard lab sample holder/analyzer, and the like.

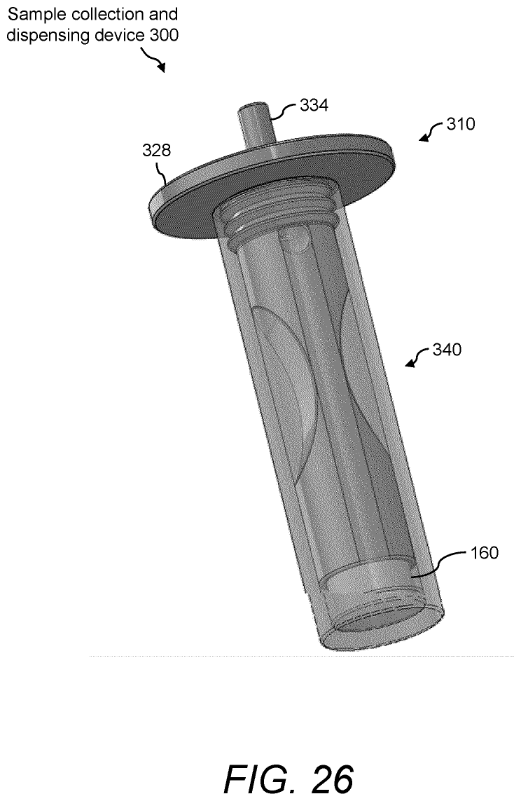

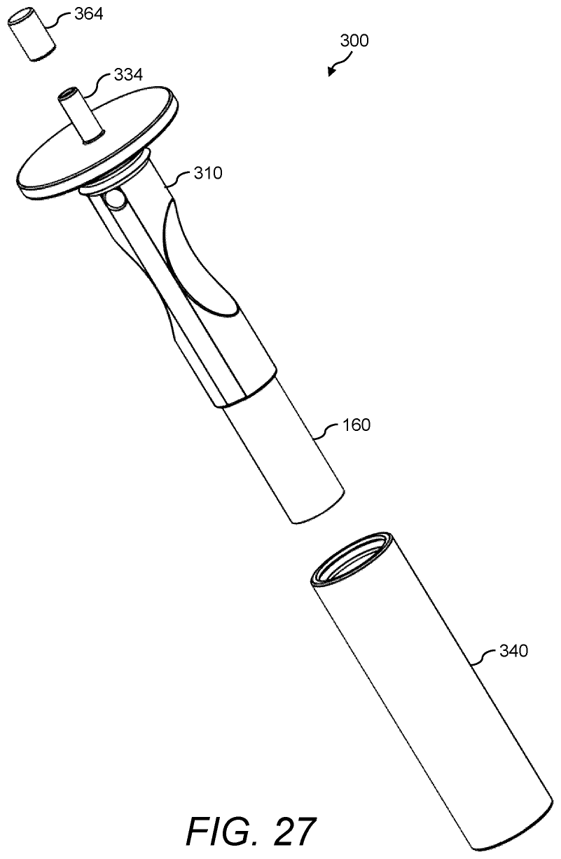

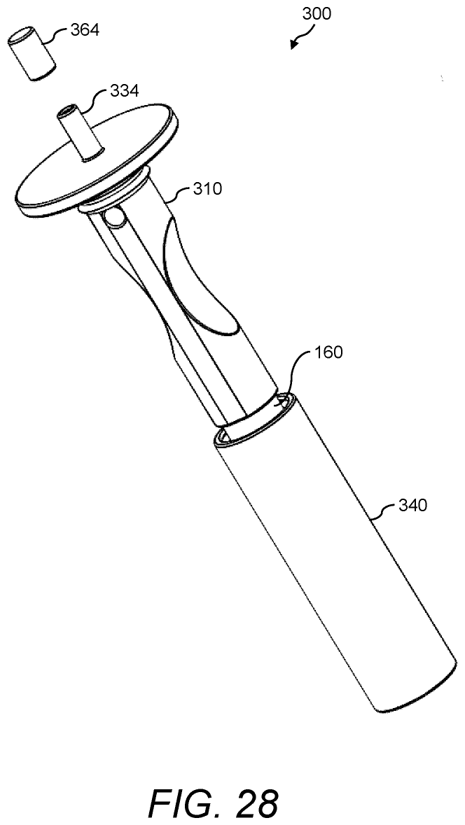

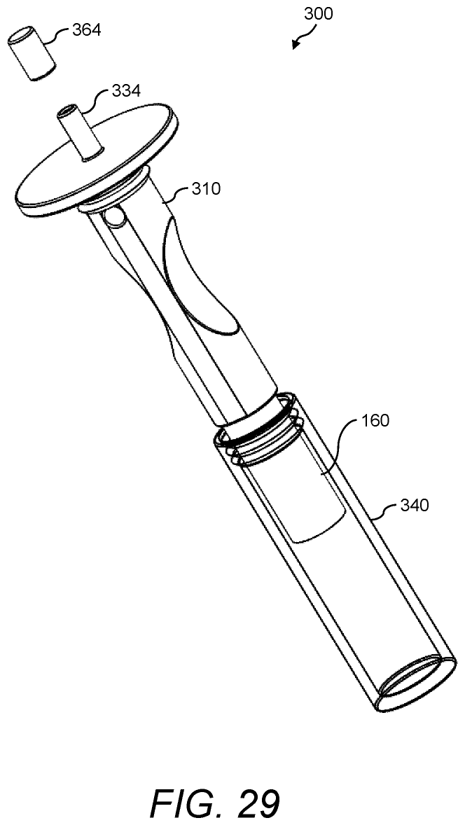

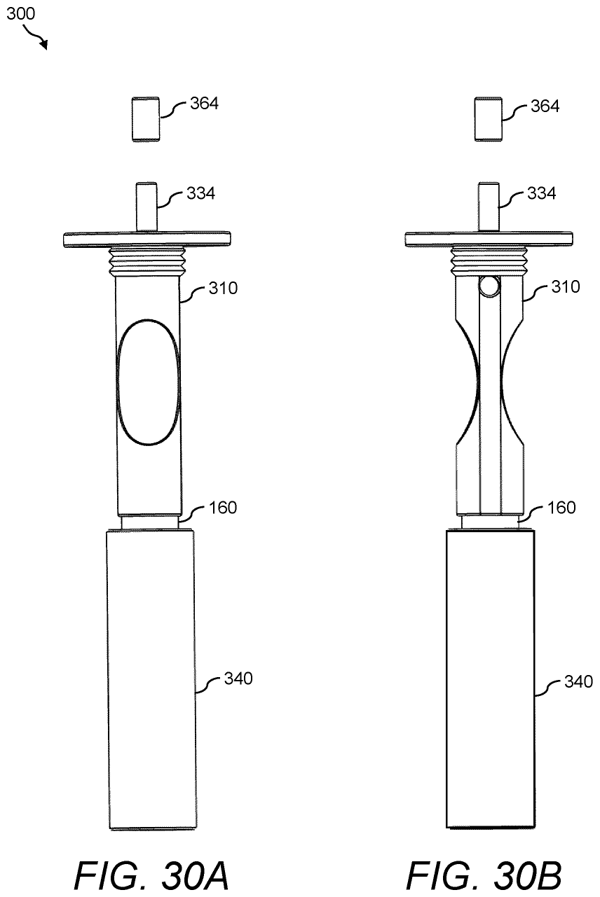

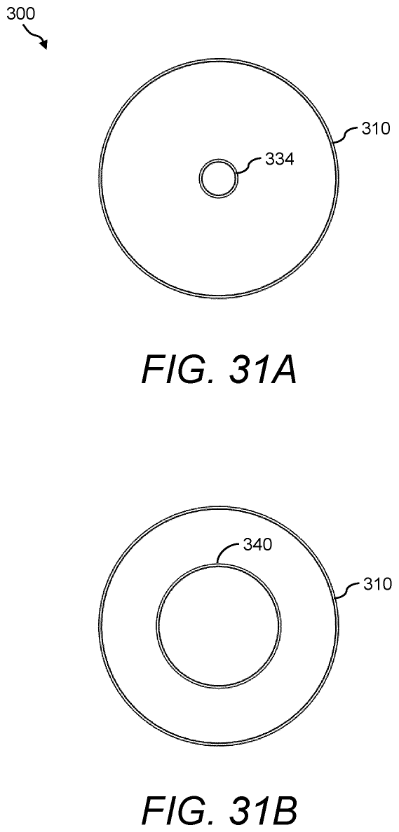

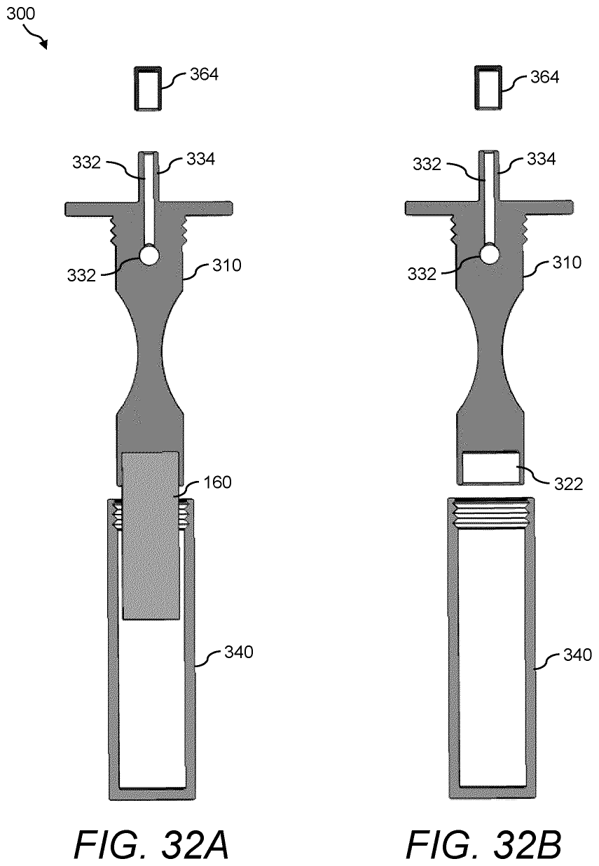

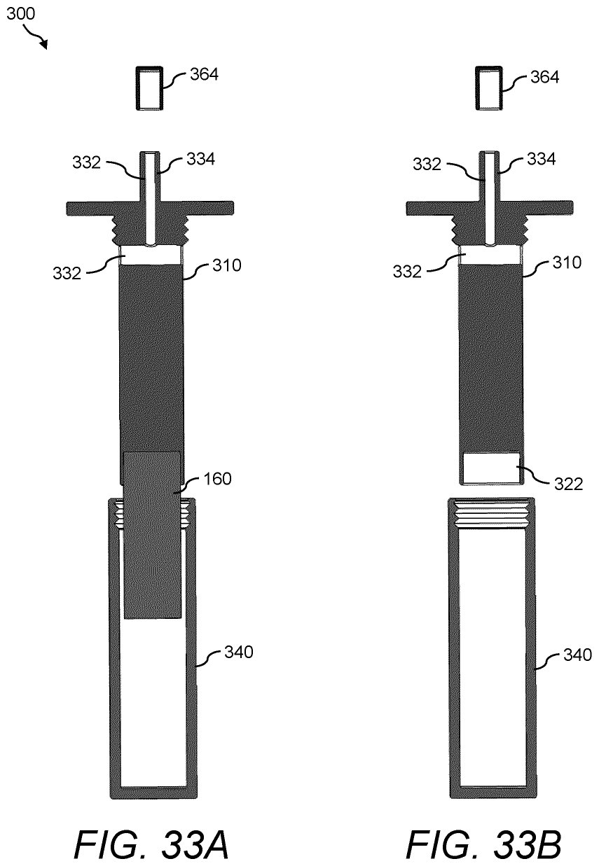

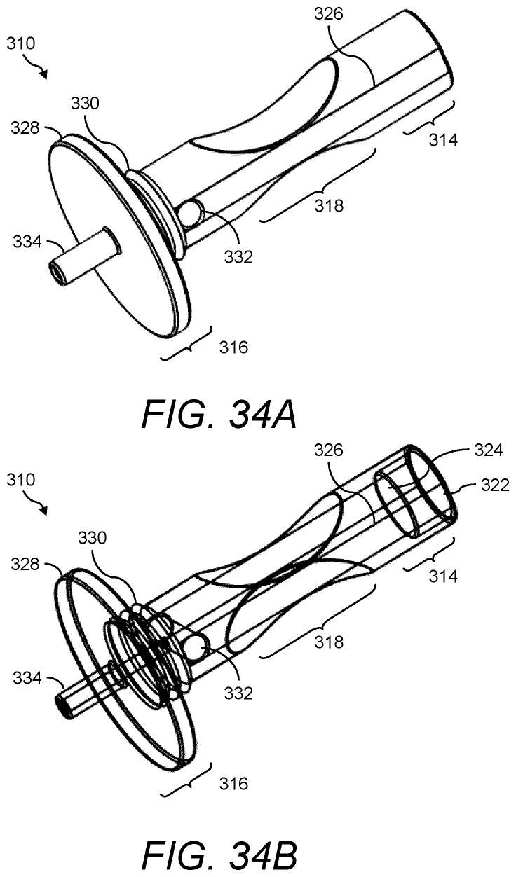

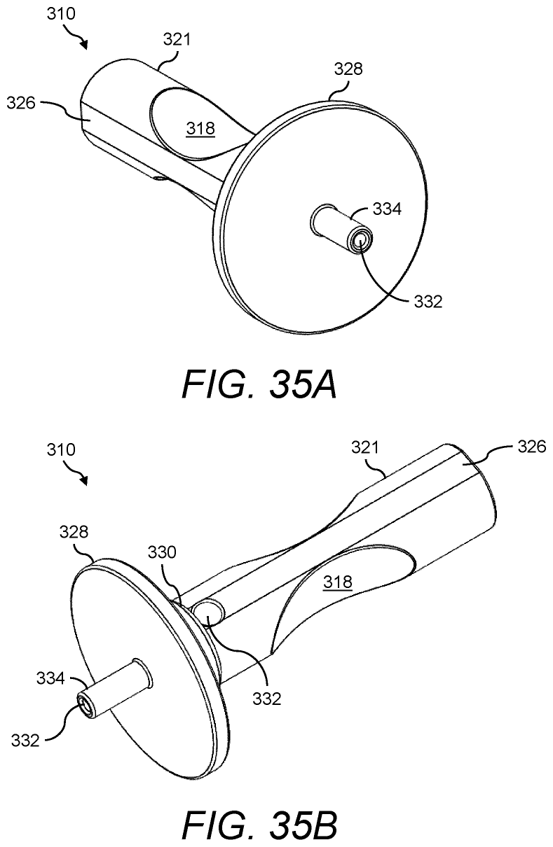

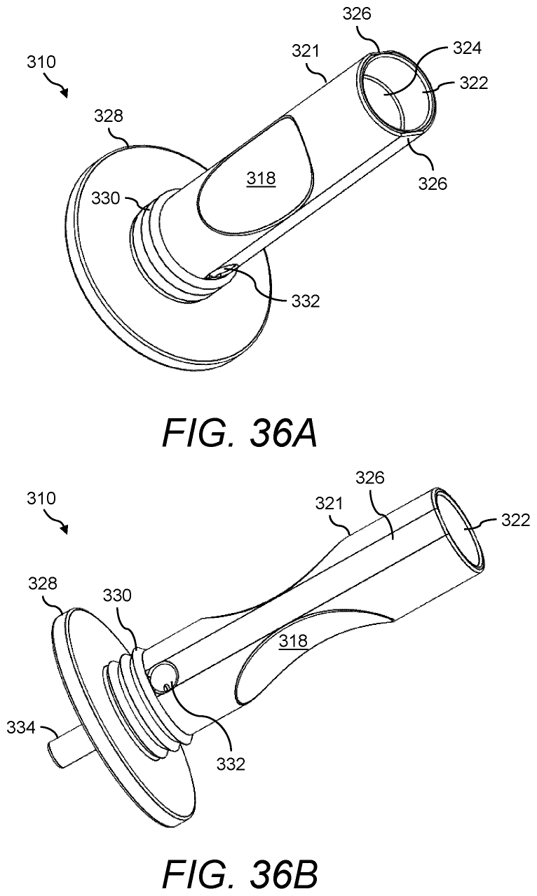

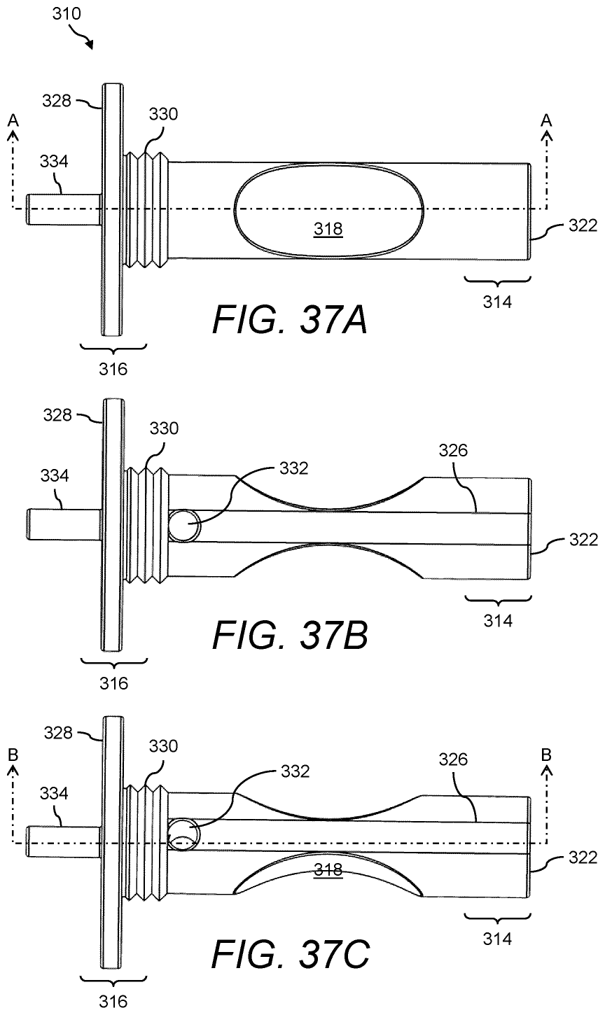

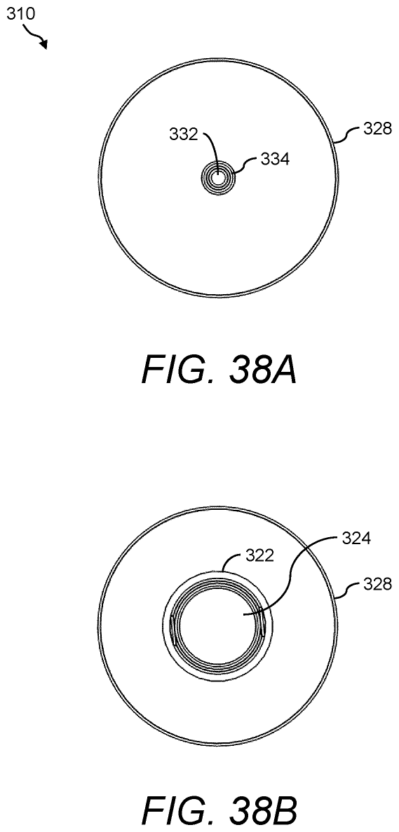

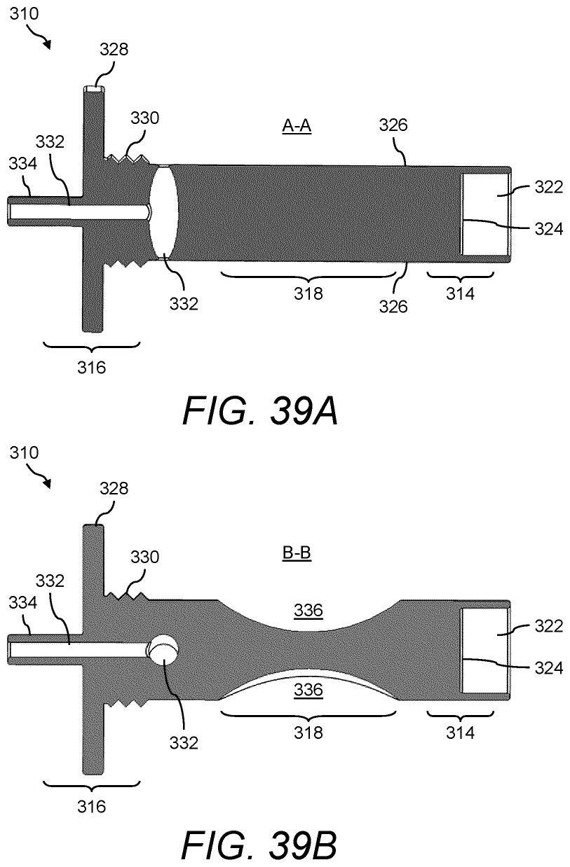

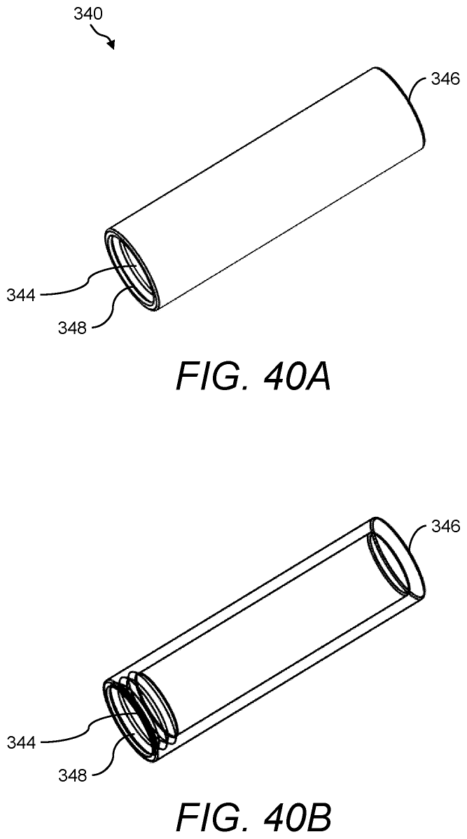

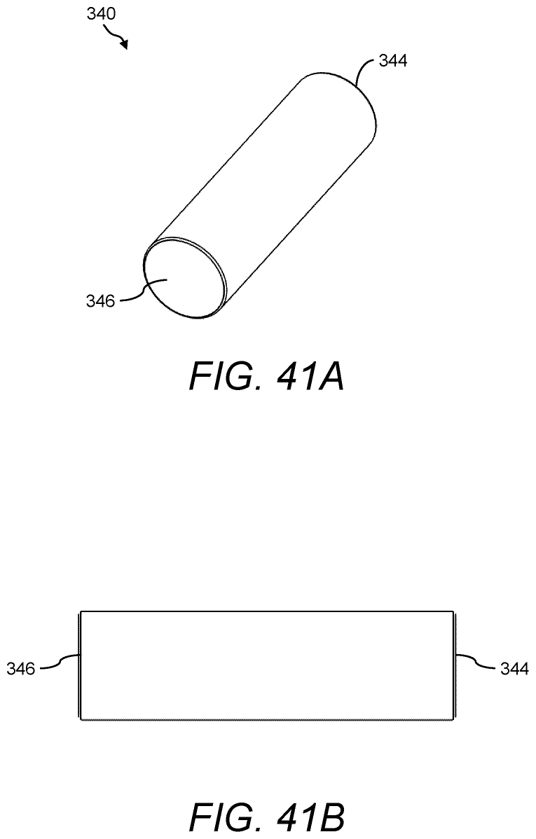

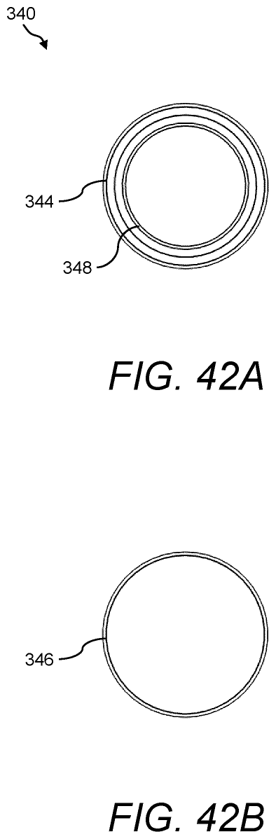

Referring now to FIG. 26 through FIG. 42B is various views of a sample collection and dispensing device 300, which is another example of the sample collection. For example, FIG. 26 shows sample collection and dispensing device 300 in a “ready to actuate” state. FIG. 27, FIG. 28, and FIG. 29 show exploded perspective views of sample collection and dispensing device 300. FIG. 30A and FIG. 30B show side views of sample collection and dispensing device 300. FIG. 31A and FIG. 31B show end views of sample collection and dispensing device 300. FIG. 32A, FIG. 32B, FIG. 33A, and FIG. 33B show various cross-sectional views of sample collection and dispensing device 300. FIG. 34A and FIG. 34B show a solid perspective view and a transparent perspective view, respectively, of an example of a plunger 310 of sample collection and dispensing device 300. FIG. 35A, FIG. 35B, FIG. 36A, and FIG. 36B show various perspective views of plunger 310 of sample collection and dispensing device 300. FIG. 37A, FIG. 37B, and FIG. 37C show various side views of plunger 310 of sample collection and dispensing device 300. FIG. 38A and FIG. 38B show end views of plunger 310 of sample collection and dispensing device 300. FIG. 39A and FIG. 39B is cross-sectional views of plunger 310 of sample collection and dispensing device 300. FIG. 40A and FIG. 40B show a solid perspective view and a transparent perspective view, respectively, of an example of a barrel 340 of sample collection and dispensing device 300. FIG. 41A and FIG. 41B show a perspective view and a side view, respectively, of an example of barrel 340 of sample collection and dispensing device 300. FIG. 42A and FIG. 42B show end views of an example of barrel 340 of sample collection and dispensing device 300.

Referring still to FIG. 26 through FIG. 42B, sample collection and dispensing device 300 may include a plunger 310 and a flexible and/or squeezable barrel 340. Plunger 310 may include one or more flow channels for directing a precise amount to sample liquid from a sample collection sponge 160 to a dispense outlet 334. Further, plunger 310 may include a narrow neck portion for providing a compressible space or void when plunger 310 is installed within the flexible and/or squeezable barrel 340.

More specifically, plunger 310 of sample collection and dispensing device 300 has a collection end 314 and a dispensing end 316. Further, a central region of plunger 310 may include a concave-shaped neck portion 318 (see FIG. 34A through FIG. 37C). Again, in some embodiments there may be only one collection space or void 136 on one side of neck portion 118 of plunger 110, instead of two collection spaces or voids 136 (one on each side). A sponge receptacle 322 may be provided at collection end 314 of plunger 310 for holding sample collection sponge 160 (as described in FIG. 1 through FIG. 23B). Sponge receptacle 322 may include a floor 324 (see FIG. 36A). Two flow channels 326 may be provided on the outside of plunger 310. For example, two flow channels 326 run along the length of plunger 310, one flow channel 326 on each side.

A plate 328 may be arranged at dispensing end 316 of plunger 310. Threads 330 may be provided at dispensing end 316 of plunger 310 and near the underside of plate 328. Another flow channel 332, which may be, for example, a T-channel may be provided at dispensing end 316 of plunger 310. For example, a T-portion of flow channel 332 is oriented toward neck portion 318 of plunger 310 and opens into the two flow channels 326 (see FIG. 36B and FIG. 39A). Then, the other end of flow channel 332 extends through plate 328 and supplies dispense outlet 334 of plunger 310.

In one example, plunger 310 may be formed of thermoplastic material using an injection molding process. Accordingly, plunger 310 may be substantially rigid and lightweight. In one example, the overall length of plunger 310 may be from about 10 mm to about 100 mm. In one example, the 10 mm-plunger 310 may be used to process small-volume samples. In another example, the 100 mm-plunger 310 may be used to process large-volume samples. Further, plunger 310 may be from about 20 mm to about 50 mm in diameter. Further, the depth of sponge receptacle 322 may be from about 2 mm to about 20 mm in one example, or from about 0.5 mm to about 2 mm in another example. Further, plate 328 may be from about 0.5 mm to about 5 mm thick. Further, plate 328 may be from about 20 mm to about 50 mm in diameter in one example, or from about 50 mm to about 100 mm in diameter in another example. Further, to mate to, for example, standard DMF devices and/or microplates, dispense outlet 334 may be from about 3 mm to about 30 mm long and from about 1 mm to about 10 mm in diameter. Further, a cap 364 may be sized to snap-fit onto dispense outlet 334.

Barrel 340 may be a cylinder-shaped barrel or reservoir that has an open end 344 and a closed end 346. In one example, barrel 340 may be fully or at least partly flexible and/or squeezable. In another example, barrel 340 may be include an element that may be fully or at least partly flexible and/or squeezable. In yet another example, barrel 340 may include an add-on component that may be fully or at least partly flexible and/or squeezable. A rim 348 may be provided around the outside of open end 344 of barrel 340 and threads 350 may be around the inside of open end 344 of barrel 340 and near rim 348. Threads 330 of plunger 310 are designed to engage with threads 350 of barrel 340 and thereby hold together plunger 310 and barrel 340 in sealed fashion. Further, when plunger 310 is inside barrel 340 there may be two spaces or voids 336 at the concave-shaped neck portion 318 of plunger 310. Together, the two flow channels 326, the two flow channels 332, and the two spaces or voids 336 may be designed to hold and dispense some desired amount of liquid in the range of from about 10 μL to about 1500 μL in one example, from about 100 μL to about 200 μL in another example, or about 150 μL in yet another example.

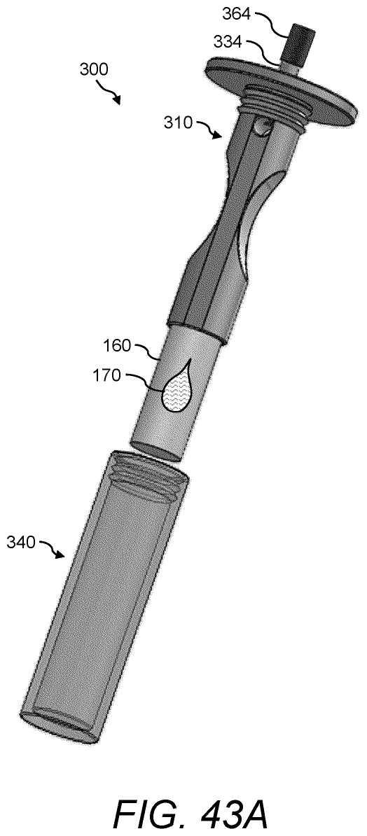

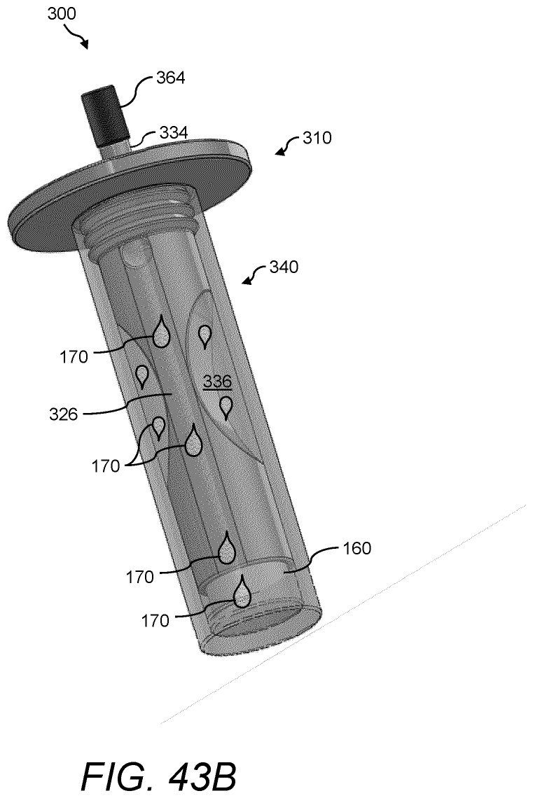

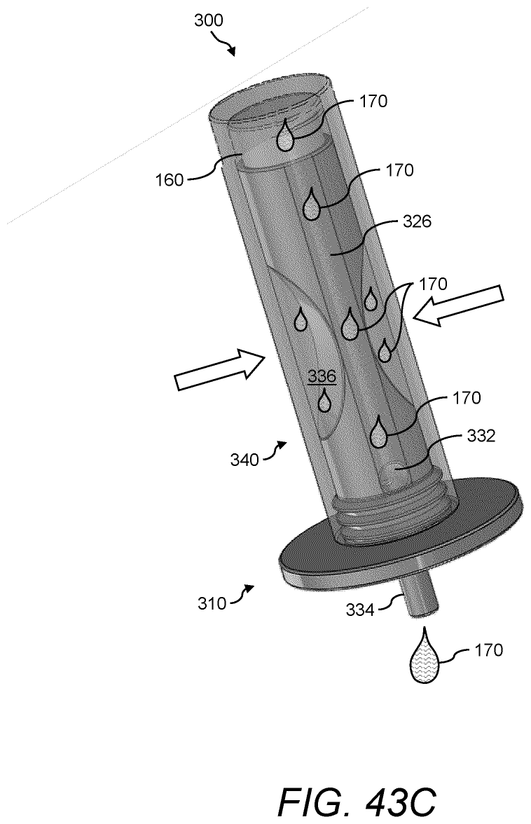

In one example, barrel 340 may be formed of thermoplastic material using an injection molding process. In one example, the walls of the cylinder-shaped barrel 340 may be formed suitably thin to provide flexibility thereof. For example, the walls of barrel 340 may be formed suitably thin to allow the sides of barrel 340 to be squeezed or compressed inward (see FIG. 43C). The dimensions of barrel 340 may vary depending on the size of plunger 310.

Sample collection and dispensing device 300 may operate substantially the same as sample collection and dispensing device 100 as described in method 250 shown in FIG. 24 and FIG. 25A through FIG. 25E. For example, FIG. 43A, FIG. 43B, and FIG. 43C is views showing an example of certain process steps of using the sample collection and dispensing device 300 shown and described hereinabove with reference to FIG. 26 through FIG. 42B.

For example, FIG. 43A shows that a user may use plunger 310 that is holding sample collection sponge 160 to collect a sample 170 (e.g., saliva) from the mouth of the subject.

Next, FIG. 43B shows that the user may insert plunger 310 holding sample collection sponge 160 into barrel 340 of sample collection and dispensing device 300. Then, the user pushes plunger 310 and sample collection sponge 160 fully into barrel 340. Then, the user screws plunger 310 and barrel 340 together in sealed fashion via threads 330 of plunger 310 and threads 350 of barrel 340. In doing so, sample collection sponge 160 is compressed and causing sample 170 (e.g., saliva) to be forced out of barrel 340 and into the two flow channels 326 along the sides of plunger 310 and into the two spaces or voids 336.

Next, FIG. 43C shows that the user may use his/her fingers to squeeze or compress the sides of barrel 340 of sample collection and dispensing device 300. More specifically, the user may use his/her fingers to squeeze or compress barrel 340 at the sides of neck portion 318 of plunger 310. In this way, the walls of barrel 340 collapse against the sides of neck portion 318 of plunger 310, which creates pressure and forces the sample 170 within the two flow channels 326 to flow into T-portion of dispense flow channel 332, then out of dispense outlet 334 of sample collection and dispensing device 300, and then into the DMF device (e.g., DMF device 200 shown in FIG. 4) or microplate (e.g., 96-well microplate). Accordingly, sample 170 may be dispensed using the sample collection and dispensing device 300. Again, sample collection and dispensing device 100 may be sized to dispense some desired amount of sample 170 (e.g., saliva) anywhere in the range of from about 10 μL to about 1500 μL in one example, from about 100 μL to about 200 μL in another example, or about 150 μL in yet another example.

The sample collection and dispensing devices 100, 300, 400 may include a filter in addition to the sample collection sponge 160. By way of example, FIG. 44A and FIG. 44B show cross-sectional views showing an example of sample collection and dispensing device 100 including a filter 190. In this example, filter 190 is fitted into sponge receptacle 122 and then sample collection sponge 160 is fitted into sponge receptacle 122. FIG. 44A shows sample collection and dispensing device 100 with both filter 190 and sample collection sponge 160 installed. FIG. 44B shows sample collection and dispensing device 100 with only filter 190 installed.

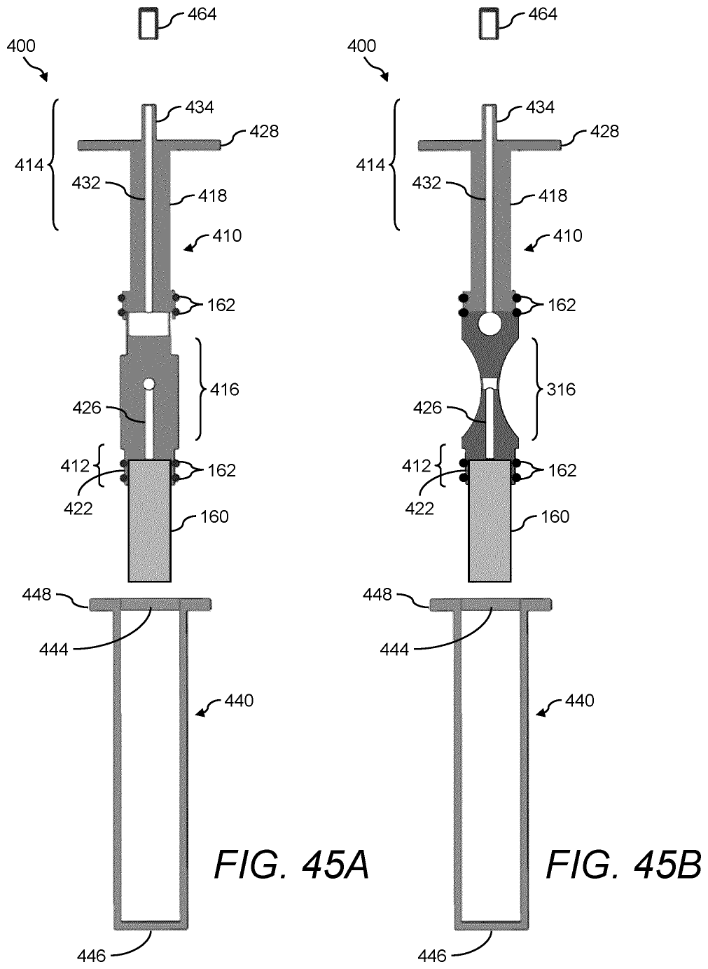

The sample collection and dispensing devices are not limited to the configurations shown hereinabove with respect to sample collection and dispensing devices 100, 300. Other configurations are possible. For example, FIG. 45A and FIG. 45B show cross-sectional views of a sample collection and dispensing device 400, which is another example of the sample collection and dispensing device. In sample collection and dispensing device 400, the plunger and barrel engage via two sets of seal rings instead of threads.

In this example, sample collection and dispensing device 400 may include a plunger 410 that is designed to be fitted into a barrel 440, a sample collection sponge 160, and a cap 464. Plunger 410 may be designed to hold sample collection sponge 160 on one end and to dispense liquid from the opposite end. For example, plunger 410 has a collection end 412, a dispensing end 414, and a concave-shaped neck portion 416. In one example, both plunger 410 and barrel 440 may be formed of thermoplastic material using an injection molding process.

Dispensing end 414 of plunger 410 may include an outlet neck 418 leading from concave-shaped neck portion 416 to a plate 428 and a dispense outlet 434. Further, a pair of seal rings 162 may be provided at the outlet neck 418-end of concave-shaped neck portion 416. Another pair of seal rings 162 may be provided at the opposite side of concave-shaped neck portion 416, toward dispensing end 414.

A sponge receptacle 422 may be provided at collection end 412 of plunger 410 for holding sample collection sponge 160. Sponge receptacle 422 supplies one end of a first stage flow channel 426, which may be, for example, a T-channel. Further, the T-portion of flow channel 426 opens to the sides of a neck portion 416 of plunger 410. Accordingly, the T-portion of flow channel 426 supplies a space or void around the neck portion 416 of plunger 410. In one example, flow channel 426 may be designed to hold any desired amount of liquid in the range of from about 10 μL to about 1500 μL.

A second stage flow channel 432, which may be, for example, a T-channel may be provided along outlet neck 418 at dispensing end 414 of plunger 410. For example, a T-portion of flow channel 432 is oriented toward neck portion 416 of plunger 410 and opens into a space or void. Then, the other end of flow channel 432 extends through outlet neck 418 and plate 428 and supplies dispense outlet 434 of plunger 410. Further, when plunger 410 is inside barrel 440 there may be two spaces or voids at the concave-shaped neck portion 416 of plunger 410. Together, the two flow channels 432, the two flow channels 436, and the two spaces or voids around the neck portion 416 of plunger 410 may be designed to hold and dispense a certain volume of liquid, such as a volume of from about 10 μL to about 1500 μL in one example, from about 100μL to about 200 μLL in another example, or may be about 150 μL in yet another example.

Barrel 440 may be the cylinder-shaped barrel or reservoir that has an open end 444 and a closed end 446. A rim 448 may be around the outside of open end 444 of barrel 440. In one example, barrel 440 may be fully or at least partly flexible and/or squeezable. In another example, barrel 440 may be include an element that may be fully or at least partly flexible and/or squeezable. In yet another example, barrel 440 may include an add-on component that may be fully or at least partly flexible and/or squeezable. For example, the thermoplastic walls of the cylinder-shaped barrel 440 may be formed suitably thin to provide flexibility that may allow the sides of barrel 440 to be squeezed or compressed inward. The dimensions of barrel 440 may vary depending on the size of plunger 410.

In sample collection and dispensing device 400, when plunger 410 and sample collection sponge 160 are pushed fully into barrel 440, both sets of seal rings 162 are engaged within barrel 440 and serving to fluidly constrain sample 170 (e.g., saliva) therein.

Sample collection and dispensing device 400 shown in FIG. 45A and FIG. 45B may operate substantially the same as sample collection and dispensing device 100 shown in FIG. 5 through FIG. 25E and/or sample collection and dispensing device 300 shown in FIG. 26 through FIG. 43C.

In summary and referring now again to FIG. 1 through FIG. 45B, the sample collection and dispensing devices 100, 300, 400 and method 250 may provide a plunger (e.g., plungers 110, 310, 410) for holding a sample collection sponge (e.g., sponge 160), a flexible and/or squeezable barrel (e.g., barrels 140, 340, 440) for receiving the plunger and sample collection sponge, and a cap (e.g., caps 164, 364, 464) for capping a dispense outlet (e.g., dispense outlets 134, 334, 434).

In some embodiments, the sample collection and dispensing devices 100, 300, 400 and method 250 may provide a plunger (e.g., plungers 110, 310, 410) capable of holding a sample collection sponge (e.g., sponge 160) on one end and including a dispense outlet (e.g., dispense outlets 134, 334, 434) at its opposite end and wherein the plunger may include one or more flow channels for directing a precise amount to sample from the sample collection sponge to the dispense outlet.

In some embodiments, the sample collection and dispensing devices 100, 300, 400 and method 250 may provide a plunger (e.g., plungers 110, 310, 410) that includes a narrow neck portion (e.g., neck portions 118, 318, 416) for providing a compressible space or void when the plunger is installed in a flexible and/or squeezable barrel (e.g., barrels 140, 340, 440).

In some embodiments, the sample collection and dispensing devices 100, 300, 400 and method 250 may provide method steps that may include, but not are not limited to, collecting a sample volume using the plunger holding the sample collection sponge, inserting the plunger holding the sample collection sponge and sample volume fully into the flexible and/or squeezable barrel and thereby causing sample liquid to flow from the sponge in the barrel (because of compressing the sponge) into the one or more flow channels in the plunger, and then squeezing the flexible barrel at the neck portion of the plunger and thereby causing a substantially precise amount of sample liquid to flow from the one or more flow channels in the plunger and out of the dispense outlet because of the displacement action of squeezing the barrel.

In some embodiments, the sample collection and dispensing devices 100, 300, 400 and method 250 may provide (1) an easy way to manually collect a sample (e.g., saliva), (2) a convenient way to store a sample (e.g., saliva), and (3) an easy way to manually dispense a precise amount of sample liquid (e.g., saliva) into, for example, a DMF device (e.g., DMF device 200 shown in FIG. 4) or microplate (e.g., 96-well microplate).

In some embodiments, the sample collection and dispensing devices 100, 300, 400 and method 250 may provide a disposable single actuation device.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “includes,” and “including” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter. For example, the term “about,” when referring to a value can be meant to encompass variations of, in some embodiments ±100%, in some embodiments ±50%, in some embodiments +20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

1. A sample collection and dispensing device, comprising:
(a) a plunger, the plunger having a linear axis, a first end, a second end and a first flow channel running along the linear axis of the plunger;
(b) a sample collection sponge, wherein the sample collection sponge is attached to the second end of the plunger; and
(c) a cylinder-shaped barrel, wherein the cylinder-shaped barrel comprises a first open end for receiving the plunger and a second closed end, and wherein the cylinder-shaped barrel comprises a flexible side wall; and
wherein the sponge is operable to collect a sample fluid;
wherein the cylinder-shaped barrel is capable of holding and storing the sample fluid; and
wherein the first flow channel is operable to dispense a fluid from the cylinder-shaped fluid reservoir.

2. The sample collection and dispensing device of claim 1, wherein the first flow channel comprises a first end and a second end, wherein the first end comprises an open dispensing outlet and the second end is connected to a first exit hole in the side of the plunger.

3. The sample collection and dispensing device of any preceding claim 1, wherein the sample collection and dispensing device further comprises a second flow channel, and wherein the second flow channel connects the second end of the plunger to a second exit hole in the side of the plunger.

4. The sample collection and dispensing device of claim 3, wherein the second exit hole comprises a T-shaped exit hole having two openings on opposite sides of the plunger.

5. The sample collection and dispensing device of claim 1, wherein the cylinder-shaped barrel has an internal volume of from about 20 μL to about 2000 μL.

6. The sample collection device and storage device of claim 1, wherein the sample collection and dispensing device further comprise a removable cap, and wherein the removable cap is attached to the first end of the plunger to cap or close the dispensing outlet of the first flow channel.

7. The sample collection and dispensing device of claim 1, wherein the plunger further comprises a circular-shaped plate at the dispensing end of the plunger and wherein the plate has a plate diameter, wherein the plate diameter is the same as, or larger than a diameter of the cylinder-shaped barrel.

8. The sample collection and dispensing device of claim 7, wherein the plate is operable to cover the cylinder-shaped barrel.

9. The sample collection and dispensing device of claim 1, wherein the plunger and the cylinder-shaped barrel further comprise threads and wherein the threads of the plunder and capable of engaging the threads of the cylinder-shaped barrel.

10. The sample collection and dispensing device of claim 9, wherein the threads on the plunger are on the underside of the circular-shaped plate and the threads on the cylinder-shaped barrel are on the inner wall of the cylinder-shaped barrel.

11. The sample collection and dispensing device of claim 10, wherein the plate further comprises a seal or a gasket.

12. The sample collection and dispensing device of claim 11, wherein seal or gasket is operable to close the open end of the cylinder-shaped barrel and optionally wherein the seal or gasket provides a fluid tight seal.

13. The sample collection and dispensing device of claim 1, wherein the plunger diameter can be adjusted relative to a diameter of the cylinder-shaped barrel to adjust the overall fluid volume of the device.

14. The sample collection and dispensing device of claim 1, wherein the plunger further comprises a concave-shaped neck portion.

15. The sample collection and dispensing device of claim 14, wherein the concave-shaped neck portion forms an internal void space between the plunger and the side wall of the cylindershaped barrel.

16. The sample collection and dispensing device of claim 15, wherein the void has a volume of from about 15 μL to about 1500 μL.

17. The sample collection and dispensing device of claim 1, wherein the second end of the plunger has a diameter smaller than a diameter of the cylinder-shaped barrel.

18. The sample collection and dispensing device of claim 17, wherein the second end of the plunger further comprises one or more seals to create a fluid tight seal between the cylinder-wall and the plunger.

19.-32. (canceled)
